(19) United States
(12) Patent Application Publication
(10) Pub. No.: US 2025/0148589 A1
(43) Pub. Date: May 8, 2025

(54) EXCREMENT ANALYSIS APPARATUS, EXCREMENT ANALYSIS METHOD, PRE-COLONOSCOPY STATE CONFIRMATION APPARATUS, STATE CONFIRMATION SYSTEM, STATE CONFIRMATION METHOD, AND NON-TRANSITORY COMPUTER-READABLE MEDIUM

(71) Applicant: NEC Platforms, Ltd., Kawasaki-shi, Kanagawa (JP)

(72) Inventors: Hiroyuki Tomishima, Kanagawa (JP); Tsutomu Miena, Kanagawa (JP); Haruhiko Yamabuchi, Kanagawa (JP); Masahiro Wakabayashi, Kanagawa (JP); Kazuhiro Kakehata, Kanagawa (JP)

(73) Assignee: NEC Platforms, Ltd., Kawasaki-shi, Kanagawa (JP)

(21) Appl. No.: 18/694,003

(22) PCT Filed: Oct. 5, 2022
(86) PCT No.: PCT/JP2022/037321

§ 371 Date: Mar. 21, 2024

(30) Foreign Application Priority Data
Oct. 28, 2021 (JP) 2021-176986

(51) Int. Cl. G06T 7/00; G06T 7/10

(52) U.S. Cl. CPC G06T 7/0012; G06T 7/10; G06T 2207/20036; G06T 2207/20081

(57) ABSTRACT

An excrement analysis apparatus being capable of dealing with a toilet bowl having various shapes and a toilet seat and capable of accurately analyzing image-captured excrement is provided. The excrement analysis apparatus inputs imaging data captured by an image capture apparatus being installed in such a way as to include, in a capturing range, an excretion range of excrement in a toilet bowl of a toilet. The excrement analysis apparatus performs classification of a capturing target substance in a pixel unit by using semantic segmentation with respect to imaging data being input. The excrement analysis apparatus outputs a classification result.

### TECHNICAL FIELD

The present disclosure relates to an excrement analysis apparatus, an excrement analysis method, a state confirmation apparatus before colonoscopy, a state confirmation system before colonoscopy, a state confirmation method before colonoscopy, and a program.

### BACKGROUND ART

A caregiver who provides excretion assistance at a caregiving site is required to reduce incontinence of a care receiver and promote support for independence of the care receiver while maintaining dignity of the care receiver. Since the excretion assistance at a caregiving site may damage dignity of a care receiver depending on occasions, a caregiver is forced to bear a heavy burden, and support for reducing a load of work is required.

In order to provide such support, a mechanism of managing excretion of a user of a toilet has been proposed by installing a sensor in the toilet and analyzing data acquired by the sensor. For example, Patent Literature 1 describes a determination apparatus that aims to reduce an increase in apparatus cost in an analysis on excrement using machine learning.

The determination apparatus described in Patent Literature 1 includes an image information acquisition unit, a preprocessing unit, an estimation unit, and a determination unit. The image information acquisition unit acquires image information on a target image being a target of determining a determination matter related to feces and being the target image acquired by capturing an image of an internal space of a toilet bowl after excretion. The preprocessing unit generates an entire image indicating the entire target image and a partial image indicating a partial region of the target image. The estimation unit inputs the entire image to a learned model that has learned, by machine learning using a neural network, a correspondence relationship between a learning entire image being an image indicating an entire internal space of the toilet bowl after excretion and a determination result of a global first determination matter among the determination matters. The estimation unit thereby performs first estimation regarding the first determination matter on the entire image. The estimation unit inputs the partial image to a learned model that has learned, by machine learning using a neural network, a correspondence relationship between a learning partial image being a partial region of the entire learning image and a second determination matter being more detailed than the first determination matter among the determination matters. The estimation unit thereby performs second estimation regarding the second determination matter on the partial image. The determination unit performs determination regarding the determination matter with respect to the target image, based on an estimation result by the estimation unit.

In addition, in colonoscopy, pretreatment in order to clean an intestine with an intestinal cleansing agent (laxative) is performed, and then an examination is performed. There are a case where the pretreatment is performed at home, followed by a hospital visit and endoscopy, and a pattern in which the pretreatment is performed in a state of being hospitalized. In case of being at home, a person performs confirmation of an effect of a cleansing agent, and in case of hospitalizing, an examiner performs the confirmation. In the examination, it is necessary that a residue is completely absent in the intestine by the cleansing agent, and particularly, when the examination is performed in a hospital, it is necessary for an examiner to confirm repeatedly, and there is a problem that a time burden and a mental burden of an examinee (medical examinee) and the examiner are imposed. In addition, there is a case where it is not possible to correctly determine by confirmation by the examinee himself/herself.

Further, in pre-examination work of colonoscopy, a case involving excretion assistance may infringe on privacy of an examinee, a mental and time burden is imposed on the examinee or an examiner, and there is a need for assistance in reducing the burden on the work. In addition, a system that particularly maintains privacy of an examinee and promotes work support is required. In some cases, an examinee performs the pre-examination work by himself/herself, but in the case involving the excretion assistance by an examiner, the examinee enters a toilet with the examiner and visually confirm excrement by observing an excretion behavior of the examinee. Observing an excretion behavior is a shame for the examinee, and also imposes a mental burden on the examiner.

In order to solve such a problem in pre-examination work, a technique in which an image acquired by capturing excrement is used for determining whether there is no problem in a time when an examinee performs endoscopy is also known. For example, Patent Literature 2 describes an endoscope operation support apparatus for improving efficiency of an operation of a medical professional related to a pretreatment for lower-part endoscopy.

The endoscope operation support apparatus described in Patent Literature 2 includes an image acquisition unit that acquires a captured image of an excretion target of a patient to which a pretreatment medicine for lower-part endoscopy is administered, and an image analysis unit that analyzes the captured image. Further, the endoscope operation support apparatus includes a determination unit that determines whether the patient is in a state where the lower-part endoscopy can be performed, based on an image analysis result, and a notification unit that notifies a terminal apparatus of a determination result via a network.

### CITATION LIST

### Patent Literature

Patent Literature 1: Japanese Unexamined Patent Application Publication No. 2020-187693
Patent Literature 2: Japanese Unexamined Patent Application Publication No. 2016-066301

### SUMMARY OF INVENTION

In the technique described in Patent Literature 1, image information of a target image acquired by capturing an internal space of a toilet bowl after excretion is acquired, a divided image including a partial region of the target image is generated, an entire image and the partial image are input to each of a learned model and another learned model, and thereby first and second estimation are performed. However, in the technique described in Patent Literature 1, since the partial region becomes a region being determined according to a shape of the toilet bowl, only the toilet bowl having a common shape can be dealt with, considering that a foreign body other than excrement is also captured. When the technique described in Patent Literature 1 is applied to a toilet bowl having a shape different from the common shape, it is difficult to perform accurate estimation.

In other words, in the technique described in Patent Literature 1, it is not possible to deal with distributed toilet bowls having various shapes, and it is necessary to construct and implement two learned models for each shape of the toilet bowl in order to deal therewith. In addition, such a problem is more complicated in consideration of reflection of a buttocks washing machine when the buttocks washing machine is attached to a toilet seat of a toilet bowl. In other words, in the technique described in Patent Literature 1, in order to perform accurate estimation by dealing with a set of a toilet bowl having various shapes and a toilet seat, it is necessary to construct and implement two learned models for each set.

Therefore, it is desired to develop an excrement analysis apparatus being capable of dealing with toilet bowls having various shapes and a toilet seat, and capable of accurately analyzing captured excrement.

Note that, in the technique described in Patent Literature 2, a ratio of pixels having black color, brown color, and intermediate color thereof to all pixels in an analysis region is detected, and when the ratio exceeds a predetermined ratio, it is determined as a state where a solid body is mixed with excrement and lower-part endoscopy cannot be performed. Therefore, the technique described in Patent Literature 2 is not intended to analyze excrement in a toilet bowl in detail, and is also not intended to improve accuracy of the excrement.

Further, in the technique described in Patent Literature 2, in order to acquire an image to be analyzed, it is necessary for a patient or a medical professional not only to manually photograph excrement in a toilet bowl with a terminal apparatus, but also to form a mark indicating a photographing range in a stagnant part in the toilet bowl. Therefore, in the technique described in Patent Literature 2, not only it takes time and effort for photographing, but also it can deal with only a dedicated toilet bowl in which a mark is formed in advance, and it is not possible to deal with various toilet bowls that are distributed. Although it is conceivable to form a mark manually after manufacturing of a toilet bowl, by seal attaching work or painting work, it is difficult to form the mark at a position where accurate determination can be made for each of the toilet bowls having various shapes, and it also takes time and effort to form the mark.

The present disclosure is made in order to solve the above-described problem, and an object thereof is to provide an excrement analysis apparatus, an excrement analysis method, a program, and the like that are capable of dealing with a toilet bowl having various shapes and a toilet seat and capable of accurately analyzing image-captured excrement.

An excrement analysis apparatus according to a first aspect of the present disclosure includes an input unit that inputs imaging data captured by an image capture apparatus being installed in such a way as to include, in a capturing range, an excretion range of excrement in a toilet bowl of a toilet. The excrement analysis apparatus includes a classification unit that performs classification of a capturing target substance in a pixel unit by using semantic segmentation with respect to imaging data being input by the input unit, and an output unit that outputs a classification result by the classification unit.

An excrement analysis method according to a second aspect of the present disclosure includes inputting imaging data captured by an image capture apparatus being installed in such a way as to include, in a capturing range, an excretion range of excrement in a toilet bowl of a toilet. The excrement analysis method includes performing classification processing of classifying a capturing target substance in a pixel unit by using semantic segmentation with respect to input imaging data, and outputting a classification result by the classification processing.

A program according to a third aspect of the present disclosure is a program for causing a computer to execute excrement analysis processing. The excrement analysis processing includes inputting imaging data captured by an image capture apparatus being installed in such a way as to include, in a capturing range, an excretion range of excrement in a toilet bowl of a toilet. The excrement analysis processing includes performing classification processing of classifying a capturing target substance in a pixel unit by using semantic segmentation with respect to input imaging data, and outputting a classification result by the classification processing.

According to the present disclosure, it is possible to provide an excrement analysis apparatus, an excrement analysis method, a program, and the like that are capable of dealing with a toilet bowl having various shapes and a toilet seat and capable of accurately analyzing image-captured excrement.

### BRIEF DESCRIPTION OF DRAWINGS

FIG. 1 is a block diagram illustrating one configuration example of an excrement analysis apparatus according to a first example embodiment;

FIG. 2 is a diagram illustrating one configuration example of an excrement analysis system according to a second example embodiment;

FIG. 3 is a block diagram illustrating one configuration example of an excrement analysis apparatus in the excrement analysis system in FIG. 2;

FIG. 4 is a schematic diagram for describing a processing example in the excrement analysis system in FIG. 2;

FIG. 5 is a diagram for describing a processing example in the excrement analysis apparatus in the excrement analysis system in FIG. 2;

FIG. 6 is a diagram for describing a processing example in the excrement analysis apparatus in the excrement analysis system in FIG. 2;

FIG. 7 is a diagram illustrating one example of a feces characteristic analysis included in the processing example in FIG. 6;

FIG. 8 is a diagram for describing a processing example in the excrement analysis apparatus in the excrement analysis system in FIG. 2;

FIG. 9 is a diagram for describing a processing example in the excrement analysis apparatus in the excrement analysis system in FIG. 2;

FIG. 10 is a flowchart for describing a processing example in the excrement analysis apparatus in the excrement analysis system in FIG. 2;

FIG. 11 is a block diagram illustrating one configuration example of an excrement analysis apparatus (a state confirmation apparatus before colonoscopy) according to a third example embodiment;

FIG. 12 is a flowchart for describing a processing example in the state confirmation apparatus in FIG. 11;

FIG. 13 is a schematic diagram for describing a processing example in a state confirmation apparatus according to a fourth example embodiment;

FIG. 14 is a diagram for describing a processing example in the state confirmation apparatus in FIG. 13;

FIG. 15 is a diagram for describing a processing example in the state confirmation apparatus in FIG. 13;

FIG. 16 is a diagram for describing a processing example in the state confirmation apparatus in FIG. 13;

FIG. 17 is a flowchart for describing a processing example in the state confirmation apparatus in FIG. 13;

FIG. 18 is a flowchart for describing a processing example in the state confirmation apparatus in FIG. 13;

FIG. 19 is a flowchart following FIG. 18;

FIG. 20 is a diagram illustrating one example of a feces color analysis included in a secondary analysis in the processing example in FIG. 18; and

FIG. 21 is a diagram illustrating one example of a hardware configuration of an apparatus.

### EXAMPLE EMBODIMENT

Hereinafter, example embodiments will be described with reference to the drawings. Note that, in the example embodiments, the same or equivalent elements may be given the same reference signs, and redundant description thereof will be omitted as appropriate. In addition, a reference sign and a name of an element in the drawings are provided to each element as one example to facilitate understanding for the sake of convenience, and are not intended to limit any content of the present disclosure in any way. In addition, there is a drawing in which a unidirectional arrow and a bidirectional arrow are drawn in the drawings described below, but any of the arrows clearly indicates a direction of a flow of a certain signal (data), and does not exclude bidirectional and unidirectional characteristics.

### First Example Embodiment

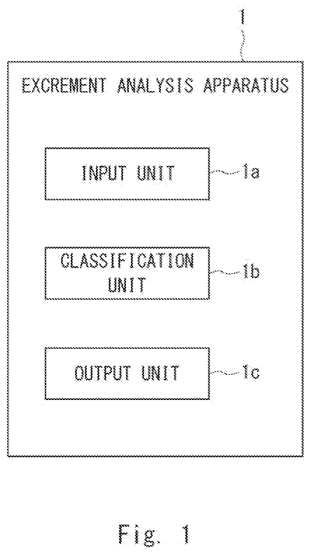

An excrement analysis apparatus according to a first example embodiment will be described with reference to FIG. 1. FIG. 1 is a block diagram illustrating one configuration example of the excrement analysis apparatus according to the first example embodiment.

As illustrated in FIG. 1, an excrement analysis apparatus 1 according to the present example embodiment can include an input unit 1a, a classification unit 1b, and an output unit 1c.

The input unit 1a inputs imaging data (image data) captured by an image capture apparatus (hereinafter, exemplified by a camera) being installed in such a way as to include, in a capturing range, an excretion range of excrement in a toilet bowl of a toilet. The imaging data are used in the excrement analysis apparatus 1 in order to analyze a content of excrement and acquire information thereof.

Thus, the camera installed in such a manner is connected to or included in the excrement analysis apparatus 1. However, it can be said that it is preferable for the excrement analysis apparatus 1 to include the camera in terms of integrating with the apparatus and preventing an outflow of imaging data to outside. The camera is not limited to a visible light camera, may be an infrared light camera and the like, and may also be a video camera as long as a still image can be extracted. When the camera is connected to the outside of the excrement analysis apparatus 1, the camera may be connected to the input unit 1a. The imaging data may include additional information (attached information) such as a capturing date and time and a capturing condition. The capturing condition may include, for example, a resolution of a camera whose resolution can be set, and a zoom magnification in a case of a camera having a zoom function.

The excretion range described above may be a region including a stagnant part of the toilet bowl, and may also be referred to as a scheduled excretion range. By installing the camera in such a way as to include such an excretion range in the capturing range, captured imaging data include excrement and the like as a subject. Of course, the excretion range described above is preferably set in such a way that a user (a person who uses a toilet, and a user of the toilet) is not reflected, and the camera is preferably installed in such a way that a lens of the camera is also not seen by the user. In addition, when a user uses the excrement analysis apparatus 1 in a hospital or a care facility, for example, the user described above is mainly a care receiver such as a patient. In addition, examples of a carer include a caregiver, also include a doctor according to occasions, but may also include a helper other than a caregiver, and may be other persons.

The classification unit 1b performs classification of a capturing target substance in a pixel unit by using semantic segmentation with respect to imaging data (analysis target data) being input by the input unit 1a. Semantic segmentation refers to a deep learning algorithm that classifies all pixels in an image and associates a label or a category with all pixels. Although the following description assumes that a label is associated with a pixel, it is also possible that a category is associated with a pixel, or a label and a category to which a plurality of labels belong are associated with a pixel. For example, examples of the semantic segmentation include, but are not limited to, a fully convolutional network (FCN), U-net, SegNet, and the like.

Note that, a pixel unit basically refers to one pixel unit, but is not limited thereto. For example, it is also possible that data acquired by performing filtering processing or the like on imaging data in preprocessing are input, and the classification unit 1b performs classification of the capturing target substance in a plurality of pixels unit in the original imaging data with respect to the input analysis target data.

The capturing target substance is a substance captured by a camera, and may include feces (also referred to as stool or excreta) depending on an installation position or an installation purpose of the camera. Therefore, the classification unit 1b performs processing of classifying a pixel into feces, for example, in a case where the pixel corresponds to feces, that is, processing of associating a label indicating the feces. As will be described later in a second example embodiment, since feces can also be classified into a plurality of feces characteristics, in a case of performing up to such classification, the classification unit 1b can perform processing of classifying a pixel into a certain feces characteristic when the pixel indicates feces and corresponds to the certain feces characteristic, that is, processing of associating a label indicating the feces characteristic. Note that, in this case, for example, it is also possible that a category “feces” is associated with a pixel, and also a label indicating a feces characteristic is associated with the pixel.

In addition, it can be supposed that the capturing target substance includes urine (pee), urine dripping, toilet paper, a buttocks washing machine, and the like. Therefore, similarly, when a pixel corresponds to urine, urine dripping, toilet paper, or a buttocks washing machine, the classification unit 1b performs processing of classifying the pixel into each of urine, urine dripping, toilet paper, or a buttocks washing machine. In other words, the classification unit 1b performs processing of associating a label or a category indicating each of urine, urine dripping, toilet paper, and a buttocks washing machine, respectively. In addition, feces and urine can also be classified for color thereof, and in such a case, a label indicating an associated feces color or a label indicating an associated urine color can be associated with the pixel. Note that, the buttocks washing machine is a device for washing a buttock, and may be referred to as a buttocks washing apparatus, a buttocks washing machine, or the like, and will be described below as a buttocks washing machine. The buttocks washing machine can be included in, for example, a hot water washing toilet seat such as Washlet (registered trademark) having a toilet flushing function.

As described above, the classification unit 1b performs classification of a capturing target substance in a pixel unit by using semantic segmentation, and by such classification, it is possible to divide an image of the capturing range for each classification (i.e., for each label). Thus, the semantic segmentation can also be referred to as an image region segmentation algorithm. Note that, the classification unit 1b can also be referred to as an analysis unit due to analyzing imaging data by performing such classification. Since the classification unit 1b can analyze the imaging data being input by the input unit 1a in real time, and more specifically, classify one piece of input image data for each region in the image by one time of processing, an analysis performed herein corresponds to a real time analysis (real time classification).

Hereinafter, information acquired from the excrement analysis apparatus 1 is also referred to as excretion information. In the present example embodiment, the excretion information includes a classification result such as a label described above as information indicating a content of the excretion. However, the excretion information may also implicitly include a shape of a region classified into each label, which is indicated as the whole of the imaging data, and can also include, in the excretion information, information separately specifying the shape of such a region (e.g., a shape of feces, or the like). In addition, the excretion information can include or add date and time information indicating a capturing date and time or an acquisition date and time of the imaging data, and additional information such as a capturing condition.

The output unit 1c outputs a classification result by the classification unit 1b, or the excretion information including the classification result. The excrement analysis apparatus 1 can include a not-illustrated communication unit as a part of the output unit 1c, and the communication unit can be configured by, for example, a wired or wireless communication interface or the like.

Regardless of a format of the classification result to be output from the output unit 1c, only a part of the classification result can be output. For example, in a case where the classification result indicates that a foreign body has been mixed, only information indicating that a foreign body has been mixed can be output as the classification result. In addition, an output destination of the classification result may be determined in advance, and the output destination is not limited to one place regardless of the specific output destination.

The output destination of the classification result can be, for example, a terminal apparatus possessed by an observer observing a user of the toilet, or the like. In this case, the classification result is output to the terminal apparatus used by an observer as notification information to the observer. The notification information can include the classification result itself, but can be only information of a predetermined content according to the classification result (e.g., excretion notification information indicating that excretion has been made, and the like). Note that, the terminal apparatus used by an observer is not limited to a terminal apparatus used by an individual observer such as a carer, and can be, for example, a terminal apparatus installed at an observation station such as a nurse station, and the terminal apparatus may function as an alert apparatus. In addition, when the output destination of the classification result is a terminal apparatus used by an observer, a direct output destination can be a server apparatus or the like being capable of receiving notification information and transferring the notification to the terminal apparatus.

In this way, the classification result can be output as notification information to an observer or the like, but can also be output to a server apparatus that collects and manages excretion information as excretion information for a carer to generate a diary for a care recipient being a user of a toilet, for example. The server apparatus can be, for example, a cloud server apparatus. The server apparatus can be installed in a facility such as a hospital in a case of the facility, and can also be installed in a private house or installed in an apartment house in a case of a private use.

The excrement analysis apparatus 1 can include a control unit (not illustrated) that controls the entire excrement analysis apparatus 1, and the control unit can include a part of the input unit 1a, the classification unit 1b, and the output unit 1c described above. The control unit can be achieved by, for example, a central processing unit (CPU), a working memory, a nonvolatile storage apparatus storing a program, and the like. The program can be a program for causing the CPU to execute processing of each of the units 1a to 1c. In addition, the imaging data being input by the input unit 1a can be temporarily stored in the storage apparatus, and can be read out at a time of classification by the classification unit 1b, but the imaging data can also be temporarily stored in another storage apparatus. In addition, the control unit provided in the excrement analysis apparatus 1 can also be achieved by, for example, an integrated circuit. A field programmable gate array (FPGA) can also be employed as the integrated circuit.

Note that, a start of classification in the classification unit 1b can be performed using, as a trigger, simple detection processing having a smaller load than that of the classification. For example, imaging data input by the input unit 1a or imaging data to be output by the input unit 1a to the classification unit 1b in a subsequent stage can be data in a case where an object is detected as a subject in the excretion range or a change such as a change in color of stagnant water is detected. The detection can be performed by a camera or the input unit 1a, for example, capturing an image at all times or at a regular interval by the camera, and using acquired imaging data. Alternatively, it is also possible to capture an image, based on a user detection result from a separately provided user detection sensor (a load sensor provided in a toilet seat, a human detecting sensor, or the like), and select the imaging data at that time as data to be output by the camera or the input unit 1a to the subsequent stage.

In addition, the excrement analysis apparatus 1 is an apparatus that analyzes a content of excrement being excreted in a toilet by classification as described above and outputs excretion information including at least a classification result, and can also be referred to as a toilet excrement analysis apparatus or an excretion information acquisition apparatus. The excrement analysis apparatus 1 can be an apparatus for functioning as a toilet sensor being an edge in an excrement analysis system (analysis system) configured on a network including a terminal apparatus of an observer, an external server apparatus, and the like.

In the excrement analysis apparatus 1 having the above-described configuration, when a range in which excrement is excreted is included as a capturing range, it is possible to accurately classify a capturing target substance and output a classification result without accurately determining an installation position of a camera or a sensor (toilet sensor) including a camera. In other words, in the excrement analysis apparatus 1, by attaching a camera and a toilet sensor to a distributed toilet bowl having various types and a distributed toilet seat, it is possible to accurately perform classification of the capturing target substance and output the classification result. Therefore, according to the excrement analysis apparatus 1 according to the present example embodiment, it is possible to deal with a toilet bowl having various shapes and a toilet seat, and it is possible to accurately analyze captured excrement.

In addition, the excrement analysis apparatus 1 does not need to transmit imaging data acquired from a camera and other image data to an outside such as a cloud, and can perform an analysis of excrement only by the excrement analysis apparatus 1 installed in a toilet, for example. In other words, it is possible to configure in such a way that all of an image and a video used for an analysis in the excrement analysis apparatus 1 are processed in the excrement analysis apparatus 1, and the image and video are not transmitted to the outside. Therefore, it can be said that the excrement analysis apparatus 1 can be configured to lead to reduce a mental burden related to privacy of a user.

In addition, according to the excrement analysis apparatus 1, it is possible to accurately collect information indicating a content of excrement excreted in a toilet bowl without having to ask a user of a toilet while considering privacy of the user of the toilet, and also to deal with a scene where an immediate notification to an observer is needed. In other words, in the excrement analysis apparatus 1, while improving installation of a sensor in a toilet for reducing a burden on excretion management in observing such as nursing care, it is possible to achieve both of consideration for privacy of a user of a toilet, and notifying and recording. The notification and recording herein are notifying of an immediate event at an observing site such as a caregiving site based on a classification result and recording of accurate information. Thus, according to the excrement analysis apparatus 1, it is possible to configure in such a way as to reduce a physical burden and a mental burden on an observer and a toilet user.

### Second Example Embodiment

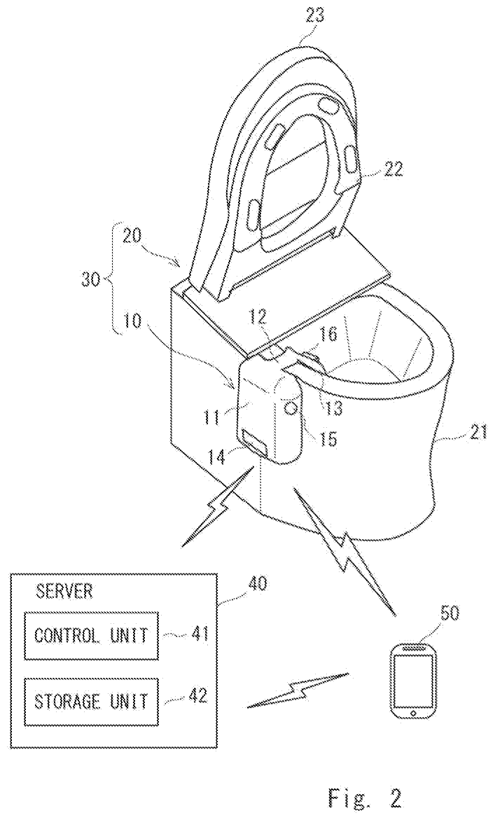

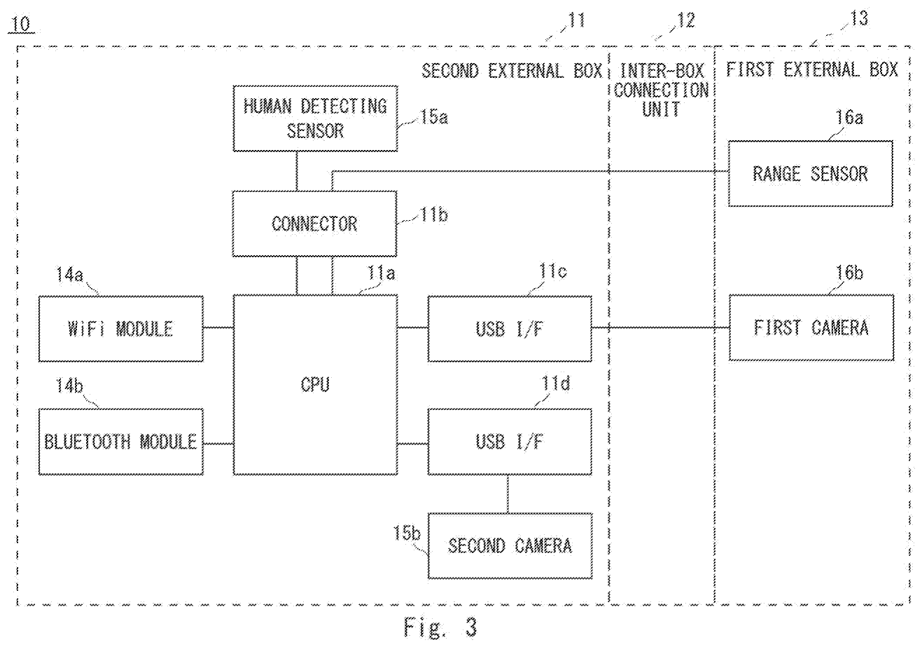

Although a second example embodiment will be mainly described with reference to FIGS. 2 to 10 while focusing on a difference with the first example embodiment, various examples described in the first example embodiment can be applied. FIG. 2 is a diagram illustrating one configuration example of an excrement analysis system according to the second example embodiment, and FIG. 3 is a block diagram illustrating one configuration example of an excrement analysis apparatus in the excrement analysis system in FIG. 2.

The excrement analysis system (hereinafter, referred to as the present system) according to the present example embodiment can include an excrement analysis apparatus 10 attached to a toilet bowl 20, a terminal apparatus 50 used by a carer, and a server apparatus (hereinafter, referred to as a server) 40. Note that, since the carer observes a user of a toilet, it can be said that the carer is one example of an observer.

The excrement analysis apparatus 10 is one example of the excrement analysis apparatus 1, and is exemplified as a toilet-bowl installation type apparatus, but may be installed in a toilet. In addition, the toilet bowl 20 can include, on a main body 21, a toilet seat 22 having a hot water washing function for user washing, and a toilet seat cover 23 for closing the toilet seat 22, for example. The excrement analysis apparatus 10 and the toilet bowl 20 can constitute a toilet bowl 30 with an analysis function having a function of outputting an analysis result including at least a classification result.

In addition, a shape of the excrement analysis apparatus 10 is not limited to a shape illustrated in FIG. 2, and can be configured such that all or some of the functions of the excrement analysis apparatus 10 are embedded in, for example, the toilet seat 22 and the like. In addition, the excrement analysis apparatus 10 can be configured in such a way that a second external box 11, which will be described later, is separated from an inter-box connection unit 12 and is disposed on a side surface side, a back surface side, or the like of the toilet bowl 20. In addition, some of the functions of the excrement analysis apparatus 10 can also be provided on the toilet seat 22 side. Note that, for example, it is also possible to adopt a configuration in which, instead of providing the excrement analysis apparatus 10 without a range sensor 16a, which will be described later, the toilet seat 22 may be provided with a weight sensor, and the excrement analysis apparatus 10 receives information from the weight sensor by wireless or wired communication. The weight sensor can also be provided in the inter-box connection unit 12, which will be described later, and can also be a pressure sensor that simply detects fixed pressure or more. In addition, it is also possible to adopt a configuration in which a first camera 16b, which will be described later, is not provided in the excrement analysis apparatus 10, a camera is provided on the toilet seat 22 side, and the excrement analysis apparatus 10 receives imaging data from the camera by wireless or wired communication.

The server apparatus (server) 40 and the terminal apparatus 50 can be wirelessly connected to the excrement analysis apparatus 10, and the terminal apparatus 50 can be wirelessly connected to the server 40. Note that, these connections can be made within one wireless local area network, for example, but it is also possible to adopt another connection form such as connection with different networks. In addition, a part or all of the connection may be made in a wired manner.

In the present system connected in such a manner, the excrement analysis apparatus 10 outputs notification information associated to a classification result by transmitting the notification information to the terminal apparatus 50, and outputs excretion information including a classification result by transmitting the excretion information to the server 40. The terminal apparatus 50 is a terminal apparatus owned by a carer of a user of the toilet, and can be a portable terminal apparatus, but may be an apparatus such as an installation-type personal computer (PC). In the former case, the terminal apparatus 50 can be a mobile phone (also including a smartphone), a tablet, a mobile PC, and the like. The server 40 can be an apparatus that collects and manages excretion information, and stores the excretion information received from the excretion analysis apparatus 10 in a viewable state from the terminal apparatus 50.

In addition, the server 40 can include a control unit 41 that controls the entire server 40, a storage unit 42 that stores excretion information in, for example, a database (DB) form, and a communication unit (not illustrated) for performing the connection as described above. The control unit 41 performs control on storing of the excretion information transmitted from the excrement analysis apparatus 10 in the storage unit 42, control on viewing from the terminal apparatus 50, and the like. The control unit 41 can be achieved by, for example, a CPU, a working memory, a nonvolatile storage apparatus storing a program, and the like. In addition, the storage apparatus can be shared with the storage unit 42, and the program can be a program for causing the CPU to achieve the function of the server 40. Note that, the control unit 41 can also be achieved by, for example, an integrated circuit.

In addition, although not illustrated, the terminal apparatus 50 can include a control unit that controls the entire terminal apparatus 50, a storage unit, and a communication unit for performing the connection as described above. Similarly to the control unit 41, the control unit can be achieved by, for example, a CPU, a working memory, a nonvolatile storage apparatus storing a program, and the like, or an integrated circuit. In addition, the program stored in the storage apparatus can be a program for causing the CPU to achieve the function of the terminal apparatus 50.

In addition, the terminal apparatus 50 preferably includes a diary generation unit that generates an excretion diary, based on the notification information received from the excrement analysis apparatus 10 and the excretion information stored in the server 40. The diary generation unit can be mounted by, for example, incorporating a diary generation application program into the terminal apparatus 50, and the like. The generated excretion diary can be stored in an internal storage unit. In addition, the diary generation unit may also be mounted as a part of a care recording unit that generates a care record. The care record generation unit can also be achieved by incorporating an application program into the terminal apparatus 50.

Next, a detailed example of the excrement analysis apparatus 10 will be described. The excrement analysis apparatus 10 can be configured by two apparatuses as illustrated in FIGS. 2 and 3, for example. More specifically, the excrement analysis apparatus 10 can include, as its housing, two boxes, for example, a first external box 13 and the second external box 11. In addition, the excrement analysis apparatus 10 can include the inter-box connection unit (inter-box connection structure) 12 that connects the first external box 13 and the second external box 11. The first external box 13 and the second external box 11 can be connected by an interface having a specific example thereof as illustrated in FIG. 3.

The excrement analysis apparatus 10 in this example can be installed on the main body 21 of the toilet bowl 20 as follows, for example. In other words, the excrement analysis apparatus 10 can be installed on the toilet bowl 20 by placing the inter-box connection unit 12 on an edge portion of the main body 21 in such a way that the first external box 13 is arranged on an inside (side where an excretion range of excrement is located) of the main body 21 and the second external box 11 is arranged on an outside of the main body 21.

For example, the range sensor 16a and the first camera 16b can be accommodated in the first external box 13. As described later, the range sensor 16a is one example of a sitting sensor that detects that sitting is performed on the toilet seat 22, and the first camera 16b is a camera that captures an image of excrement and is a camera that acquires imaging data being input by an input unit 1a in FIG. 1.

The second external box 11 includes a device that performs a real time analysis based on imaging data (image data) captured by the first camera 16b. In addition, the second external box 11 includes a communication device 14 that, under control of the device, notifies a carer and transmits an analysis result to the server 40 when an event occurs.

For example, a CPU 11a, a connector 11b, USB I/Fs 11c and 11d, a WiFi module 14a, a Bluetooth module 14b, a human detecting sensor 15a, and the second camera 15b can be accommodated in the second external box 11. Note that, USB is an abbreviation for universal serial bus, and USB, WiFi, and Bluetooth are all registered trademarks (the same applies hereinafter). The communication device 14 is exemplified by each of the modules 14a and 14b, and the CPU 11a executes a real time analysis while transmitting and receiving data to and from another portion via each of the elements 11b, 11c, and 11d as necessary. Note that, in this example, description is given on an assumption that the CPU 11a also includes a memory for temporarily storing imaging data. In addition, the communication device 14 is not limited to a communication module of the exemplified standard, and may be wireless or wired. Examples of the communication module include, for example, various modules such as a long term evolution (LTE) communication module, a fifth-generation mobile communication module, and a low power, wide area (LPWA) communication module.

As illustrated in FIG. 3, the first external box 13 and the second external box 11 are connected by an interface exemplified by the connector 11b and the USB I/F 11c, the connection line is provided inside the inter-box connecting unit 12, and thereby one excrement analysis apparatus 10 is configured.

The first external box 13 will be described.

The range sensor 16a is a sensor that measures a distance to a target object (buttocks of a user of the toilet bowl 20) and detects that a user is sitting on the toilet seat 22, and detects that the target object is sitting on the toilet seat 22 when a certain time elapses beyond a threshold value. In addition, when there is a change in the distance to the target object after sitting, the range sensor 16a detects that the user has left the toilet seat 22.

As the range sensor 16a, for example, an infrared sensor, an ultrasonic sensor, an optical sensor, and the like can be adopted. When the range sensor 16a adopts an optical sensor, it is sufficient that a transmission/reception element is disposed in such a way that light (not limited to visible light) can be transmitted and received from a hole provided in the first external box 13. In the transmission/reception element herein, a transmission element and a reception element may be configured separately, or may be integrated with each other. The range sensor 16a is connected to the CPU 11a via the connector 11b, and can transmit a detection result to the CPU 11a side.

The first camera 16b is one example of a camera that captures imaging data being input to the input unit 1a in FIG. 1, and can be an optical camera whose lens portion is disposed in the hole provided in the first external box 13. As described in the first example embodiment, the first camera 16b is installed in such a way as to include, in a capturing range, an excretion range of excrement in the toilet bowl 20 of the toilet. The first camera 16b is connected to the CPU 11a via the USB I/F 11c, and transmits the imaging data to the CPU 11a side.

The second external box 11 will be described.

The CPU 11a is an example of a main control unit of the excrement analysis apparatus 10, and controls the entire excrement analysis apparatus 10. As described later, the CPU 11a performs a real time analysis. The connector 11b connects the human detecting sensor 15a and the CPU 11a, and connects the range sensor 16a and the CPU 11a. The USB I/F 11c connects the first camera 16b and the CPU 11a, and the USB I/F 11d connects the second camera 15b and the CPU 11a.

The human detecting sensor 15a is a sensor that detects presence of a person (entry and exit of a person) in a specific region (measurement region range of the human detecting sensor 15a), and the specific region can be a region in which an entry and exit to and from a toilet can be determined. As the human detecting sensor 15a, for example, an infrared sensor, an ultrasonic sensor, an optical sensor, and the like can be adopted regardless of a detection method. The human detecting sensor 15a is connected to the CPU 11a via the connector 11b, and transmits a detection result to the CPU 11a when a person is detected in the specific region.

The CPU 11a can control an operation of the range sensor 16a and an operation of the first camera 16b, based on the detection result. For example, the CPU 11a can also perform processing of operating the range sensor 16a when the detection result indicates an entry, operating the first camera 16b when the range sensor 16b detects sitting, and the like.

The second camera 15b can be an optical camera whose lens portion is disposed in a hole provided in the second external box 11, and is an example of a camera that acquires face image data by capturing a face image of a user of the toilet in order to identify the user. The second camera 15b may be installed on the toilet bowl 20 in such a way as to include a face of a user in a capturing range, but may also be installed in a toilet room where the toilet bowl 20 is installed.

The Bluetooth module 14b is one example of a receiver that receives identification data for identifying a user from a Bluetooth tag held by the user, and can also be replaced with a module based on another near-field communication standard. The Bluetooth tag held by the user can be set as a different ID for each user, and can be held by the user, for example, by being embedded in a wristband and the like.

The WiFi module 14a is one example of a communication device that transmits various types of data including notification information to the terminal apparatus 50 and transmits various types of data including excretion information to the server 40, and can also be replaced with a module adopting another communication standard. The face image data acquired by the second camera 15b and the identification data acquired by the Bluetooth module 14b can be added to or embedded in the notification information and the excretion information, and thus can be transmitted to the terminal apparatus 50 and the server 40, respectively. The terminal apparatus 50 and the server 40 that receives the face image data can perform face authentication processing, based on the face image data, and identify the user. However, the excrement analysis apparatus 10 can also be configured in such a way as not to transmit the face image data, and, in that case, user identification by face authentication can be achieved by causing the CPU 11a to perform the face authentication processing, and identification data indicating a result of the user identification can be set as a target of transmission.

The USB I/F 11c, or the CPU 11a and the USB I/F 11c can be one example of the input unit 1a in FIG. 1, and input imaging data captured by the first camera 16b. The CPU 11a and the WiFi module 14a can be one example of the classification unit 1b in FIG. 1. The CPU 11a can perform a real time analysis on the imaging data, and perform transmission of the notification information to the terminal apparatus 50 and transmission of the excretion information to the server 40 via the WiFi module 14a. In the real time analysis, as described with respect to the classification unit 1b, classification of a capturing target substance is performed in a pixel unit by using semantic segmentation.

In addition, the notification information and the excretion information can also be transmitted via the Bluetooth module 14b and the like. In this way, each of the notification information and the excretion information can be transmitted to the terminal apparatus 50 and the server 40 respectively connected to the excrement analysis apparatus 10 via a network or a near-field wireless communication network. Of course, the transmission of the notification information may be transmission via the server 40 or another server as long as transfer to the terminal apparatus 50 is performed. It is assumed that each of the notification information and the excretion information to be transmitted are information associated to a classification result and information including the classification result, and do not include imaging data themselves. Thus, not only a mental burden related to privacy of a user can be reduced, but also an amount of transmission data can also be reduced. Particularly, it is beneficial to reduce an amount of data in an environment with poor network bandwidth. Note that, additional information (capturing date and time, and the like) of the imaging data may be transmitted including in the notification information and the excretion information.

Note that, a smartphone is illustrated as an example of the terminal apparatus 50. However, a notification destination (transmission destination) may be, for example, a notification apparatus of a nurse call system, another terminal apparatus owned by a carer, an intercom (intercommunication), and the like, in addition to or instead of the smartphone. Examples of the another terminal apparatus include, for example, a personal handy-phone system (PHS) and the like.

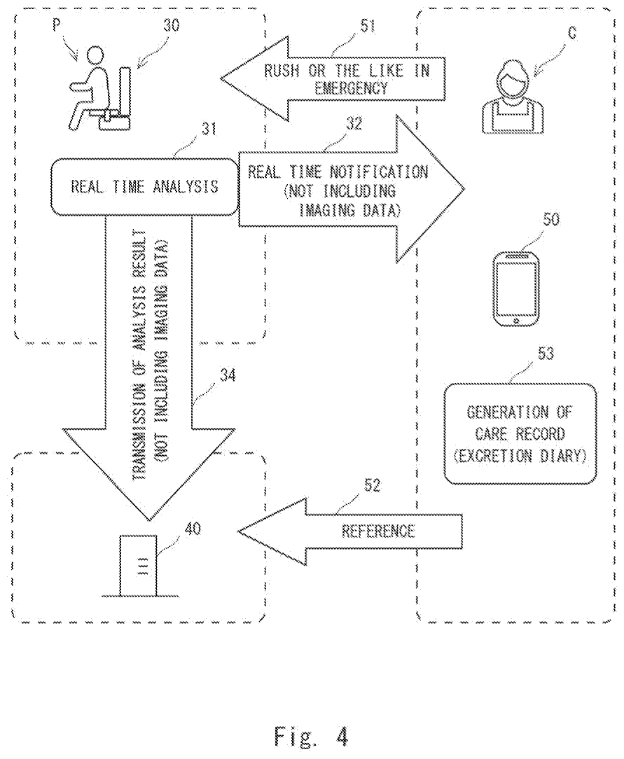

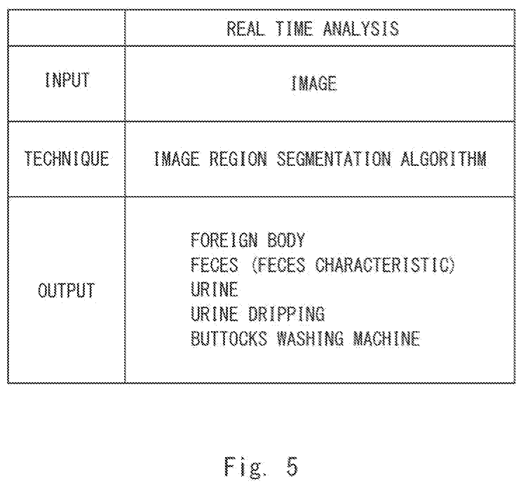

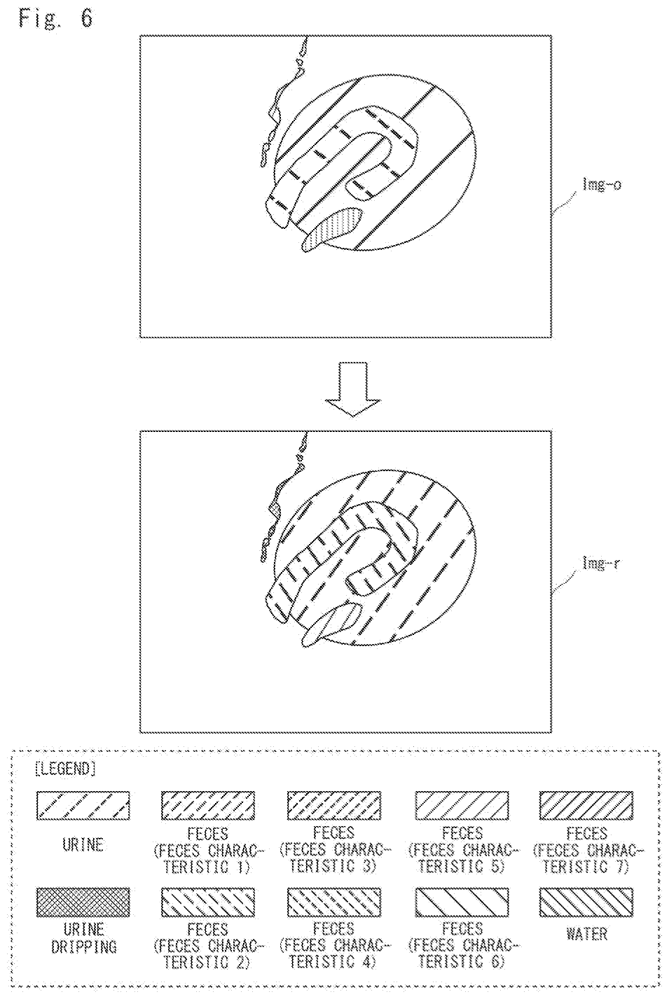

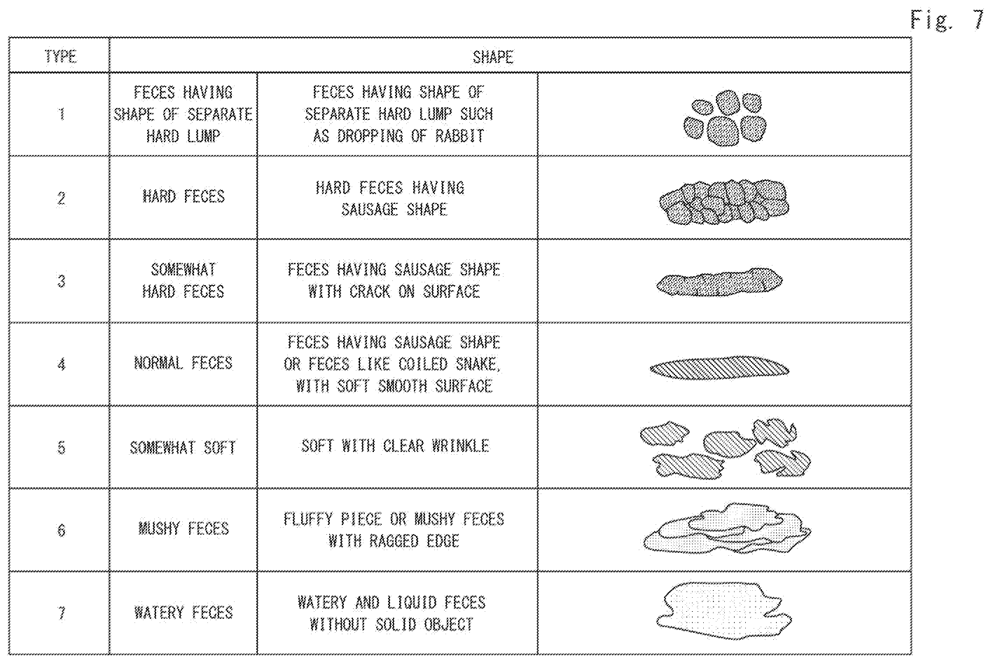

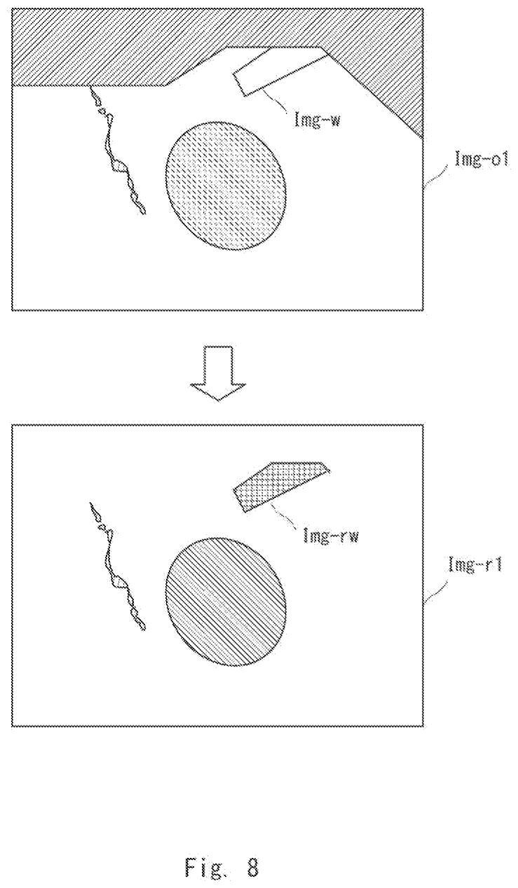

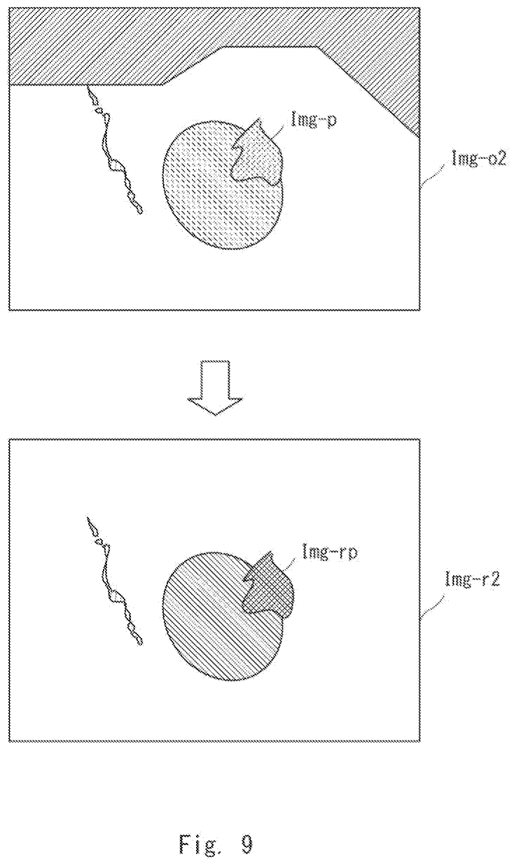

With reference to FIGS. 4 to 9, a real time analysis will be described. FIG. 4 is a schematic diagram for describing a processing example in the present system, and FIGS. 5 to 9 are diagrams for describing a processing example in the excrement analysis apparatus 10. Herein, FIG. 6 is a diagram illustrating one example of a classification image, FIG. 7 is a diagram illustrating one example of a feces characteristic analysis (feces characteristic classification) included in the processing example in FIG. 6, and FIGS. 8 and 9 are diagrams illustrating another example of a classification image.

As illustrated in FIG. 4, an example in which a user P uses the toilet bowl 30 with the analysis function being installed in a toilet, and a carer C of the user P observes a state thereof is given. When the user P uses the toilet bowl 30 with the analysis function, the CPU 11a detects that the user sits on the toilet seat, based on a detection result from the range sensor 16a functioning as a sitting sensor. When detecting sitting, the CPU 11a instructs the first camera 16b to start capturing, and performs a real time analysis 31, based on captured imaging data.

As the real time analysis 31, the CPU 11a can perform classification of a capturing target substance in a pixel unit by using semantic segmentation, and acquire a classification result. The number of classifications (the number of labels) is not limited. For example, the CPU 11a can classify, for each pixel, the capturing target substance into any of excrement, a foreign body, and another substance. In addition, the CPU 11a can also classify excrement into any of feces, urine, and urine dripping, or any of feces, urine, feces and urine (feces+urine), and urine dripping. In other words, the CPU 11a can classify, for each pixel, the capturing target substance into any of feces, urine, urine dripping, a foreign body, and another substance, or any of feces, urine, feces+urine, urine dripping, a foreign body, and another substance.

Herein, the foreign body can refer to a substance not being allowed to be discarded into the toilet bowl 20. The foreign body may be liquid or solid, and may include, for example, any one or a plurality of a urine absorbing pad, a diaper, a toilet paper core, and the like. In other words, when a pixel is labeled as a substance constituting such an object, it means that a foreign body exists.

In addition, it assumed that the another substance includes at least one of a buttocks washing machine, toilet paper, and a substance (in some cases, water only) after flushing excrement. The another substance can be classified as one label, but can also be classified into three labels, for example, a label indicating a buttocks washing machine, a label indicating toilet paper, and a label indicating a substance after flushing excrement.

In addition, the foreign body can be defined as a substance other than feces and urine as a subject except for a toilet bowl and washing liquid for the toilet bowl. When the definition is used, the foreign body may be liquid or solid as long as it is other than feces and urine, and may include, for example, any one or a plurality of a urine absorbing pad, a diaper, and a toilet paper core. In addition, the foreign body or the another substance can include, for example, any one or a plurality of vomit, melena, and blood vomiting (hematemesis).

Note that, the foreign body and the another substance need not to be overlapped with each other in the definition, are not limited to a way of distinguishing as in the above-described example, and a method of distinguishing may be determined by, for example, a type of notification to the carer C. Of course, any of the substances exemplified for the foreign body and the another substance can be classified as an individual substance label, not as a label for the foreign body or the another substance.

In addition, the CPU 11a can also perform together with at least one of classification of feces into a plurality of predetermined feces characteristics, classification of feces into a plurality of predetermined feces colors, and classification of urine into a plurality of predetermined urine colors. Herein, the feces characteristic can indicate a shape or form of feces, and for example, classification exemplified by Bristol stool scale 1 to 7 can be adopted.

Then, in a case where an immediate notification to the carer due to foreign body detection and the like needs as a result of the real time analysis 31, the CPU 11a transmits notification information (a real time notification 32) to the terminal apparatus 50 of the carer C located at a place away from the toilet via the WiFi module 14a. In this way, the CPU 11a can transmit, to the terminal apparatus 50, foreign body information (foreign body information indicating a foreign body determination result) indicating whether a foreign body is included. The foreign body information is output as at least a part of the notification information, and the CPU 11a can perform determination (foreign body determination) whether a foreign body is included depending on, for example, whether there is a pixel labeled as the foreign body (or whether there is a predetermined number of pixels). Regardless of the foreign body, in what scene the notification information is output can be set in advance, and it can also be configured in such a way that the setting can also change from the terminal apparatus 50 and the like. For example, when a classification result is classified as excrement, the CPU 11a can output an excretion notification to an observer with respect to the terminal apparatus 50 and the like.

In addition, the another substance can include at least a buttocks washing machine. Then, when the classification result of a pixel is classified as a buttocks washing machine, or when a predetermined number or more of pixels classified as a buttocks washing machine are continuously present, the CPU 11a can stop subsequent classification processing and output an excretion completion notification to an observer. The subsequent classification processing can be, for example, classification processing for the next pixel or notification processing other than another excretion completion notification. In this way, the excrement analysis apparatus 10 can be configured in such a way as to detect an end of excretion by finding a buttocks washing machine. With such a configuration, it is possible to eliminate a possibility of mixing with washing water from the buttocks washing machine, such as subsequent dripping of moisture, and thereby decreasing accuracy of the classification result. In addition, by adopting a configuration being capable of accurately classifying the buttocks washing machine in such imaging data, it is possible not only to accurately perform excretion completion notification to a carer, but also to eliminate erroneous detection such as determining, as urine, dripping of a washing liquid during detection of the buttocks washing machine.

By the notification as described above, the carer C can be released from a situation where the carer C constantly stays with the user P during excretion of the user P, and can also take a measure 51 by rushing and the like in case of emergency by the real time notification 32. Herein, the real time notification 32 to be transmitted does not include imaging data.

In addition, the CPU 11a performs a transmission 34 of a real time analysis result to the server 40 via the WiFi module 14a with respect to the excretion information including a result (classification result) of the real time analysis 31. In this way, an analysis result of the real time analysis 31 is transmitted to the server 40 by performing the analysis result transmission 34 by the communication function. The analysis result transmission 34 is transmitted without including imaging data. The information recorded in the server 40 can be set as a target of reference 52 for generation 53 of a care record (excretion diary) by the carer C and future care support.

In addition, the carer C of the user P performs the generation 53 of the care record (excretion diary) of the user P while appropriately performing the reference 52 to the excretion information of the user P being stored in the server 40, based on the received notification information, in the terminal apparatus 50. The excretion diary can be generated as a part of the care record. In this way, the terminal apparatus 50 can record the excretion diary for each user. Note that, a format and the like of the excretion diary are not limited.

In addition, the CPU 11a can output the classification result as information including a classification image being drawn by performing color classification for each classification (for each label). Such a classification image can be output to the terminal apparatus 50 as notification information or as a part of notification information, or can be output as excretion information or as a part of excretion information for generation of an excretion diary later. An example of the classification image will be described later with reference to FIG. 6.

In addition, the CPU 11a can perform classification in stages. For example, when there is a substance being classified as excrement, the CPU 11a outputs an excretion notification to the terminal apparatus 50 and the like. After output of the excretion notification, the CPU 11a can classify each pixel classified as excrement into any of feces, urine, and urine dripping, or any of feces, urine, feces+urine, and urine dripping, and also perform detailed classification. The detailed classification herein can include at least one of classification of feces into a plurality of predetermined feces characteristics, classification of feces into a plurality of predetermined feces colors, and classification of urine into a plurality of predetermined urine colors.

Herein, with reference to FIG. 5, one example of an input, a technique, and an output of the real time analysis 31 will be described including a classification example thereof. The real time analysis is an analysis that requires real time property, such as a notification to the carer C. For the real time analysis, data (imaging data) of an image captured by the first camera 16b can be an input, and be classified into any of the following five kinds by deep learning (DL), and a classification result can be an output. Herein, semantic segmentation (image region segmentation algorithm) is used as the DL. The classification result can be associated with a label associated to the kind. The five kinds exemplified herein are a foreign body (a diaper, a urine absorbing pad, and the like), feces (feces characteristic), urine, urine dripping, and a buttocks washing machine, and when the feces characteristic is classified into eight kinds, they are classified into 12 kinds in total. The classification kinds are examples of a phenomenon being a trigger of a real time notification. Note that, for example, when it is classified into the buttocks washing machine, it can be determined that excretion is completed. In addition, classification that can be determined as having completed excretion as in a case of the buttocks washing machine can include toilet paper (or toilet paper of a predetermined amount or more) or a substance after excrement has been made, and can be included in the classification.

In addition, the DL can input learning data with a correct answer label as correct answer data (training data) and cause machine learning. A learning model (i.e., a learned model) being generated as a result can be stored inside of the CPU 11a or in a storage apparatus accessible from the CPU 11a. In the real time analysis to be performed at a time of operation, imaging data are input to such a learned model (specifically, input for each piece of image data, such as for each video frame), and a classification result is acquired. In other words, the real time analysis is a comparison with learned image data. In addition, a learned model used in the real time analysis may be plural, and for example, a different learned model can be used between at least one kind of the six kinds described above and the other kinds. Note that, an algorithm of the learned model (an algorithm of machine learning) may be any algorithm belonging to the semantic segmentation, and a hyper parameter and the like such as the number of layers are not limited.

An example of the classification image described above will be described with reference to FIG. 6. An image Img-o illustrated in FIG. 6 is one piece of imaging data acquired by a camera. The CPU 11a classifies each pixel of the input image Img-o into urine, urine dripping, feces (feces characteristic 1), feces (feces characteristic 2), feces (feces characteristic 3), feces (feces characteristic 4), feces (feces characteristic 5), feces (feces characteristic 6), feces (feces characteristic 7), water, a buttocks washing machine, and a foreign body as illustrated in a legend in FIG. 6. Then, a classification image Img-r can be generated as a classification result. Generation of the classification image Img-r can be acquired by applying a color associated to a label classified into each pixel in such a way as to associate to the image Img-o. It can be seen that the classification image Img-r becomes an image in which a region is divided for each classification.

The above-described classification example of the feces characteristic will be described with reference to FIG. 7. The feces characteristic can be performed classification in a manner, for example, conforming to the Bristle stool scale illustrated in FIG. 7, and as a result of the classification, the feces characteristic can be classified into any of types 1 to 7 as illustrated in FIG. 7. “Water” in the legend in FIG. 6 may be equivalent to a type 7.

In addition, the classification image may be an image such as the example illustrated in FIG. 8 or the example illustrated in FIG. 9. As illustrated in FIG. 8, when an input image Img-o1 includes a pixel group Img-w indicating the buttocks washing machine, a classification image Img-r1 classifies a region Img-rw of the buttocks washing machine as being different from excrement and the like. In addition, as illustrated in FIG. 9, when an input image Img-o2 includes a pixel group Img-p indicating paper (toilet paper), a classification image Img-r2 classifies a region Img-rp of the paper as being different from excrement and the like. Note that, in the images Img-o1 and Img-o2, a portion represented by an oblique line with rising to a right is a portion acquired by performing a process (hereinafter, referred to as a mask process) of darkening an input image in a case where a human body portion is detected or as a non-analysis target as default.

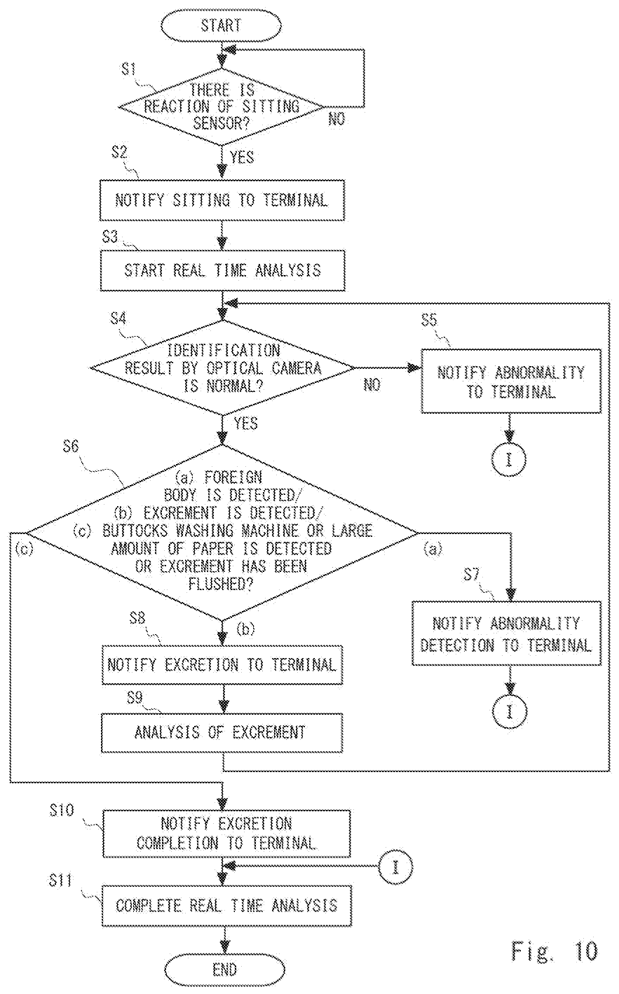

Next, one example of a procedure of real time analysis processing will be described with reference to FIG. 10. FIG. 10 is a flowchart for describing a processing example in the excrement analysis apparatus 10, and is a flowchart illustrating one example of an operation content of a real time analysis in which a user entering a toilet and sitting on a toilet seat is a trigger. The operation content described herein can be mainly performed by the CPU 11a while the CPU 11a mainly controls each unit. In addition, although a processing example using two learned models to which semantic segmentation is applied is described herein, only one of two may be a model to which semantic segmentation is applied. In addition, it is also possible to use only one learned model or use three or more learned models.

First, whether there is a reaction of the range sensor 16a functioning as a sitting sensor is checked (step S1). When there is no reaction in step S1 (in a case of NO), a reaction of the sitting sensor is waited. When a user sits, the range sensor 16a reacts, and YES is determined in step S1. When YES is determined in step S1, sitting is notified to the terminal apparatus 50 (step S2), and real time analysis also starts (step S3). Note that, when an entry is detected by the human detecting sensor 15a before sitting, the entry can also be notified to the terminal apparatus 50, and the same also applies to an exit.

In the real time analysis, an optical camera (exemplified by the first camera 16b) performs capturing inside a toilet bowl, and first, whether the acquired imaging data (e.g., the image Img-o in FIG. 6) can be normally identified is determined (step S4). Whether it can be normally identified can be determined whether there is an image being normally capable of classification, and although a determination criterion is not limited, it can be determined that, for example, an image being totally reflected or an image not being in focus cannot be normally identified. When an abnormality is detected (in a case of NO in step S4), an abnormality notification is transmitted to the terminal apparatus 50 of a carer (step S5). In this way, even when the inside of the toilet bowl cannot be normally captured, it is preferable that notification information indicating the fact is transmitted to the terminal apparatus 50. On the other hand, when the inside of the toilet bowl can be normally identified (in a case of YES in step S4), classification is performed (step S6).

In step S6, classification as to whether each pixel of an image corresponds to any of a foreign body, excrement, a buttocks washing machine, paper (toilet paper), and a substance after flushing excrement is performed by using a learned model for performing the classification. Further, in step S6, it is determined from the classification result whether a detection target object corresponds to any of (a) a foreign body, (b) excrement, and (c) a buttocks washing machine or paper (or paper of a predetermined amount or more) or a substance after excrement has been made. Herein, it is possible to determine which of (a), (b), and (c) the detection target object corresponds to, for example, by acquiring the image Img-r in FIG. 6 from a classification result for each pixel. For example, determination as to whether there is paper of a predetermined amount or more can also be performed as determination as to whether paper has an area equal to or larger than a predetermined amount, based on the area of a classified region. In addition, a learned model can be constructed in such a way as to perform such determination.

When the foreign body is detected in step S6, a foreign body detection notification is made to the terminal apparatus 50 of the carer (step S7). When the excrement is detected, an excretion notification (transmission of notification information indicating that excretion has been made) is made to the terminal apparatus 50 of the carer (step S8), and an excrement analysis is also performed (step S9). The excrement analysis is classification of excrement in a pixel unit using a learned model for performing ten kinds of classification illustrated in FIG. 6 for excrement. The learned model is also a model using semantic segmentation. By the excrement analysis, ten kinds of classification illustrated in the legend in FIG. 6 are performed for each pixel, and the image Img-r in FIG. 6 can be acquired. After the processing in step S9, the processing returns to step S4, and processing for a next image is performed.

When the detection target object detected in step S6 corresponds to the above (c), it is determined that the excretion is completed, and an excretion completion notification (transmission of notification information indicating that excretion is completed) is made to the terminal apparatus 50 of the carer (step S10). Upon completion of the processing in step S10, the real time analysis ends (step S11). In addition, the excretion completion notification may be transmitted only after a point in time when there is no reaction of the sitting sensor. The reason is that the buttocks washing machine may be used for twice or more. Note that, the real time analysis also ends after step S5 and after step S7.

In this way, in the processing example in FIG. 10, foreign body detection is always performed, and when the foreign body is detected, the carer is notified, and thereafter, capturing is started at timing of sitting, and determination of feces (feces characteristic), urine, and urine dripping as illustrated in FIG. 6 is performed with respect to an image captured at a fixed period. When feces (feces characteristic), urine, or urine dripping is detected, a preset label is associated and the classification is completed. In addition, detection of (c) described above, such as a buttocks washing machine, is also performed, and at timing when any of (c) described above is detected, an excretion completion notification is made to the terminal apparatus 50, and the determination of feces (feces characteristic), urine, and urine dripping ends. In this way, by notifying a carer and the like of start and completion of excretion, contamination with a foreign body, and the like, the carer and the like can acquire the information in real time, and thus a physical and mental burden can be reduced.

In addition, transmission timing of the excretion information to the server 40 is not limited, and for example, the excretion information may be transmitted after completion of an analysis in step S11, or may be transmitted after the processing in step S9 and before returning to step S4.

As described above, the excrement analysis apparatus 10 can acquire an excretion start, foreign body detection, excrement detection, and excretion completion as a real time analysis result, and can also acquire detailed excretion information such as feces characteristic. Any of the analysis results can be recorded in the server 40 on a cloud in a viewable state from the terminal apparatus 50, and it can also be configured in such a way as to be transmitted to the terminal apparatus 50. In addition, the server 40 can also be configured in such a way as to accumulate the received analysis result, perform a further analysis from the accumulated data, and then be able to notify the terminal apparatus 50 of the analysis results or view from the terminal apparatus 50.

In addition, the excrement analysis apparatus 10 or the present system including the excrement analysis apparatus 10 can be used in a private house on an assumption that a user is one person, but preferably has a function of identifying a user on an assumption that a plurality of users are present. As a result, it can be suitably used in a private house of a plurality of users or a facility such as a hospital and a care facility. Note that, the function is as described by using face image data acquired by the second camera 15b or identification data acquired by the Bluetooth module 14b. As a result, it is possible to notify a carer of an entry notification, an exit notification, a sitting notification, a leaving notification, an excretion start notification, an excretion completion notification, and the like together with a user name, record excretion information for each user, and generate an excretion diary and a care record including the excretion diary. In addition, although the description is given herein on an assumption that a user of a toilet is a person, the present invention can also be applied to an animal kept by a person.

Herein, an excretion diary and a care record including the excretion diary will be supplementarily described. Information acquired by the real time analysis can be used when a carer generates an excretion diary and the like of a user. In addition, a program of the terminal apparatus 50 can be executably incorporated in the terminal apparatus 50 as care software including a presentation function of presenting notification information received from the excrement analysis apparatus 10. In addition, the care software may have a function of automatically inputting, to an excretion diary or a care record including the excretion diary, information transferred from the server 40 or information acquired when the server 40 is accessed. In addition, such care software may be provided on the server 40, and, in that case, the care software may receive notification information and excretion information from the excrement analysis apparatus 10, and may automatically input the information to an excretion diary or a care record.

As described above, the present system can achieve an effect described in the first example embodiment. Particularly or in addition to the effect, the present system achieves, for example, the following effect.

The first effect is a point that, since classification can be performed for each region in an image, unlike image classification (hereinafter, image classification according to a comparative example) in which only one classification can be performed for one image, classification can be performed even when a plurality of objects are captured in the image. The first effect also cites a point that, since it is also possible to classify, for each region, excrement (in a case where there are a plurality of small objects) being difficult for object detection and divided into plural, it is possible to classify feces, urine, urine dripping, and a foreign body with high accuracy. Hereinafter, the object detection is referred to as object detection according to the comparative example. In addition, the first effect also cited that, even when a plurality of objects overlap with each other, it is possible to perform classification from a non-overlapped region of the object, and since the plurality of objects are not collectively classified into one, it is also possible to accurately classify.

Particularly, in order to achieve a notification to a carer at a time of a start of excretion, completion of excretion, and in abnormal and an accurate recode of excretion management, it is necessary to accurately detect excrement, a foreign body, and a buttocks washing machine from an image capturing an inside of a toilet bowl. An analysis using a cloud server enables an advanced analysis, but since imaging data is transmitted to the cloud server, a mental burden on privacy of a user is lead to an increase. In addition, in that case, it may take a long time for an analysis result to come out depending on a network environment due to transmission of the imaging data. Therefore, from a viewpoint of privacy protection and consideration of the network environment, it is desirable that the excrement analysis is performed by an edge device corresponding to a so-called edge of a communication network.

However, when the excrement analysis is performed in real time by the edge device, considering that a CPU having space saving and energy saving is used, since a processing capacity is low, it is conceivable to achieve the excrement analysis by image classification according to the comparative example. However, in this case, there is a problem of accuracy, and a problem that, when a plurality of objects are captured in an image, accurate classification cannot be performed by the image classification due to classifying the entire image into one label.

In addition, it is also conceivable to adopt the object detection according to a comparative example, which can be classified more accurately than the image classification according to the comparative example. When an object is detected in an image, the object detection according to the comparative example arranges a rectangle (bounding box) surrounding a detected object, and classifies an object in the bounding box. Thus, even when a plurality of objects are captured in the image, each object can be surrounded by a bounding box and classified. However, when there are a plurality of small objects, when a plurality of objects overlap with each other, when a plurality of objects are surrounded by a single bounding box, or the like, it may not be possible to accurately classify a target object according to accuracy of the bounding box surrounding the object. Further, in a case of the object detection according to the comparative example, there is a possibility that accurate object detection is not performed from imaging data due to influence that structure in a toilet bowl or a reflected image differ depending on a manufacturer or a type of the toilet bowl or a toilet seat. Particularly for a buttocks washing machine, when it can be detected from imaging data, it becomes an important determination element that can notify a carer of completion of excretion, but there is a possibility that accurate object detection cannot be performed since there is a difference depending on the manufacturer or the type of the toilet bowl or the toilet seat. In this way, even when the object detection according to the comparative example is adopted, there is a problem such as a case where accuracy of classification is poor and influence of the structure inside the toilet bowl is taken.

In contrast, in the present example embodiment, classification is performed in a pixel unit by using semantic segmentation, and thus these problems are solved, and the above-described first effect is achieved. In other words, in the present example embodiment, while improving installation of a sensor in a toilet for reducing a burden on excretion management in nursing care, it is possible to improve accuracy of an excrement analysis related to a notification to a carer, an excretion record, and the like. Then, since reliability of an analysis result is increased by the first effect, a burden on the carer can be reduced, and it can be said that thick support to a user is possible.

A second effect is a point that, by performing classification of feces by labels including feces characteristic (e.g., Bristol stool scale 1 to 7), classification including accurate feces characteristic determination can be performed in single processing, and accuracy of an analysis of excrement can be improved. Then, the second effect can reduce a burden on a carer, and it can be said that thick support to a user is possible.

Particularly, by being able to classify by labels including a feces characteristic, even when a plurality of feces characteristics can be confirmed in excrement in imaging data, accurate classification becomes possible. Further, when there is a difference in feces characteristic between a start and nearly end of excretion, it can be used for assessment for an appropriate measure, and excretion management can be easily performed. In addition, since the feces characteristic determination can be performed in single processing together with division of a region of an image, that is, at a time of classification, a real time analysis can be performed.

A third effect is a point that, in the present example embodiment, since classification for each region is performed as a result of the classification for each pixel unit, influence in difference in imaging data in a toilet bowl due to a difference in a manufacturer and a type of the toilet bowl and a toilet seat is not taken. Further, since the effect does not become a factor that deteriorates accuracy by machine learning (a factor that hinders use of a learned model), and the machine learning can be applied, an effect of having high accuracy is achieved.

A fourth effect is a point that, by classifying for each region, since it is possible to accurately perform determination of not only excrement but also a buttocks washing machine, and it is possible to distinguish dripping of washing water during detection of the buttocks washing machine from urine, it is possible to accurately determine completion of excretion and accurately notify a carer.

### Third Example Embodiment

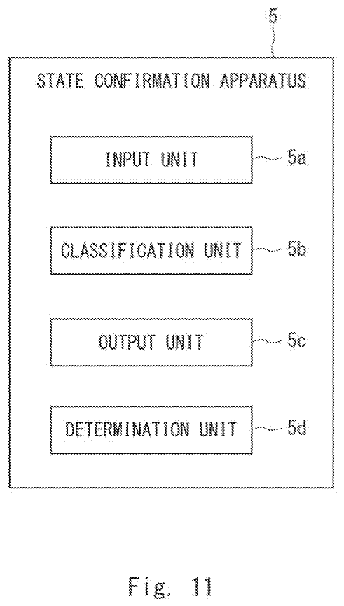

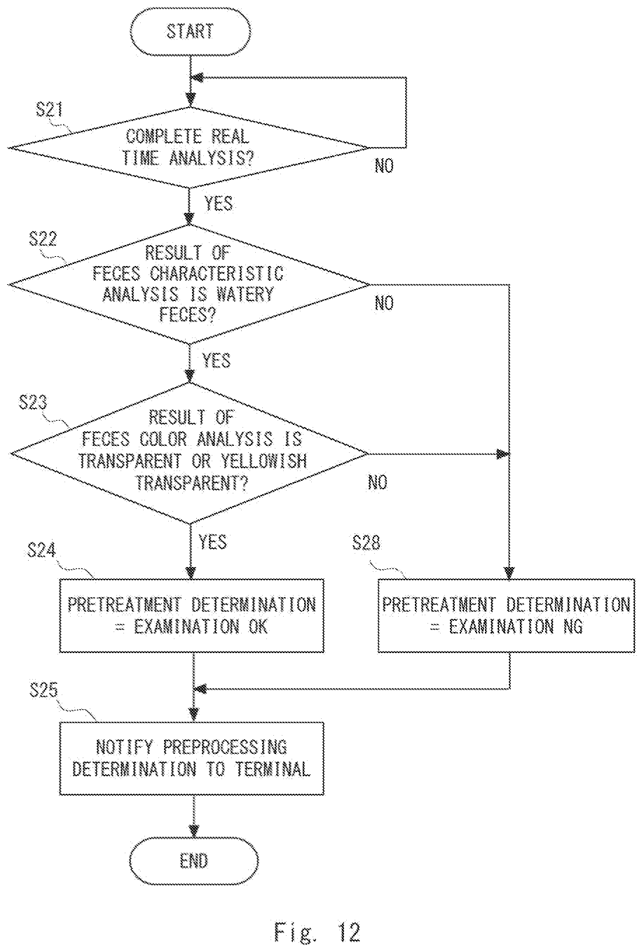

In a third example embodiment, a function for confirming a state before colonoscopy will be described with reference to FIGS. 11 and 12 with respect to an excrement analysis apparatus incorporated in the excrement analysis apparatus according to the first or second example embodiment, and the processing thereof. An excrement analysis apparatus according to the present example embodiment may be referred to as a state confirmation apparatus before colonoscopy or a colonoscopy timing determination apparatus. Although the present example embodiment is mainly described with respect to a difference from the second example embodiment, various examples described in the first and second example embodiments can be applied. FIG. 11 is a block diagram illustrating one configuration example of the excrement analysis apparatus (state confirmation apparatus before colonoscopy) according to the present example embodiment.

As illustrated in FIG. 11, the state confirmation apparatus (hereinafter, simply a state confirmation apparatus) 5 before colonoscopy according to the present example embodiment includes an input unit 5a, a classification unit 5b, and an output unit 5c being equivalent to the input unit 1a, the classification unit 1b, and the output unit 1c in FIG. 1, respectively. In addition, the state confirmation apparatus 5 can be incorporated in a system illustrated in FIG. 2 as in the second example embodiment, and thus will be described with reference to FIGS. 2 and 3 as well. In addition, the state confirmation apparatus 5 can include a control unit (not illustrated) that controls the entire state confirmation apparatus 5 and a communication unit (not illustrated), and the control unit can include a part of the input unit 5a, the classification unit 5b, and the output unit 5c described above, and a determination unit 5d (, and a calculation unit to be described later).

However, a content output by the output unit 5c is different from a content output by the output unit 1c as described later. An output destination of the output unit 5c can be basically a terminal apparatus 50 of a staff of colonoscopy, a terminal apparatus of an examinee, or a server 40. However, it is assumed that the server 40 can transfer information to the terminal apparatus 50 of the staff or the terminal apparatus of the examinee, or stores the information in such a way that the information can be viewed from the terminal apparatus 50 or the terminal apparatus of the examinee.

Further, the state confirmation apparatus 5 according to the present example embodiment includes the determination unit 5d. The determination unit 5d determines whether a user of a toilet has completed pretreatment before colonoscopy, based on a classification result by the classification unit 5b. Although a determination criterion is not limited, a criterion needs to be such that it is basically possible to determine that the pretreatment has been completed, and for example, when a feces characteristic is watery feces and a feces color is transparent or yellowish transparent, it is determined that the pretreatment has been completed.

In order to enable such determination, the classification unit 5b according to the present example embodiment also performs classification of feces as excrement into a plurality of predetermined feces characteristics and classification of feces into a plurality of predetermined feces colors. Then, the output unit 5c outputs a determination result by the determination unit 5d as a classification result in the classification unit 5b or as a part of the classification result in the classification unit 5b. The output destination can be set in advance, for example, the terminal apparatus 50 of a colonoscopy staff or the terminal apparatus of an examinee. The colonoscopy staff is an examiner, and a doctor or a nurse corresponds to the examiner. Note that, the terminal apparatus of the examinee can be a portable terminal apparatus such as a mobile phone (also including a smartphone), a tablet, or a mobile PC, but even an apparatus such as an installation-type PC has no problem when the determination result is viewed at home or the like.

Since the state confirmation apparatus 5 according to the present example embodiment can output such a determination result, a burden on an examinee (a medical examinee) and an examiner can be reduced.

In addition, the state confirmation apparatus 5 can also include a calculation unit (not illustrated) that calculates a feces amount being an amount of feces, based on a classification result by the classification unit 5b. The feces amount can be calculated, for example, by acquiring a classification image Img-r in FIG. 6, as a total area of regions classified as feces, or as the total area occupied within a certain size of an object (region classified as feces) in the classification image Img-r. Note that, the calculation may be estimation. In this case, the determination unit 5d determines whether a user of a toilet has completed pretreatment, based on the classification result by the classification unit 5b and the feces amount calculated by the calculation unit. Particularly, it is preferable to calculate the feces amount, based on the classification result using a last image at timing before flushing.

In addition, the state confirmation apparatus 5 according to the present example embodiment can be configured in such a way as to exclude the determination unit 5d, include the determination unit 5d on a server 40 side, and output a classification result to the server 40, that is, can be configured as a system in which functions are distributed to a plurality of apparatuses. Note that, the classification result may be output as a classification image, but may not be a classification result constructed as an image. In other words, in the configuration, the server 40 has a function of automatically performing determination as to whether pretreatment before colonoscopy has been completed by using a pre-stored determination database or the like. The server 40 can acquire a determination result by giving a received classification result to the function. The above-described function can be incorporated into the server 40 as a program. Even in such a configuration, in the present example embodiment, a burden on an examinee and an examiner can be reduced. In addition, even when the state confirmation apparatus 5 is configured as a single apparatus or a distributed system, when at least an optical camera and a communication device for acquiring imaging data is installed in a toilet at home, the following effect can be achieved. That is, the state confirmation apparatus 5 having such a configuration achieves an effect that at least one of an examinee and an examiner can know a determination result while the examinee is at home.

Further, in the present example embodiment, when an image capture apparatus and a communication device, for example, such as an optical camera and the communication device, are installed on a toilet bowl side, it is also possible to adopt a configuration in such a way that another piece of processing is performed on the server 40 side.

Next, a processing example of the state confirmation apparatus 5 in FIG. 11 will be described with reference to FIG. 12. FIG. 12 is a flowchart for describing a processing example in the state confirmation apparatus 5 in FIG. 11. An operation content described herein can be mainly performed by a CPU 11a in FIG. 3 while the CPU 11a mainly controls each unit. Note that, also in a configuration example in which some functions are provided on the server 40 side, only transmission/reception of information is added and a main body of the operation is changed in some operations, and processing is basically similar to the following processing example.

Hereinafter, processing after a real time analysis is performed and a classification result is acquired by the processing illustrated in FIG. 10, for example, will be mainly described. First, whether the real time analysis is completed is checked (step S21). When it is determined in step S21 that the real time analysis is not completed (in a case of NO), the processing waits until completion. When completed (in a case of YES in step S21), the state confirmation apparatus 5 determines whether an analysis result (classification result) of a feces characteristic is watery feces (e.g., “feces characteristic 7” in the legend in FIG. 6, or “water” in which a ratio of feces is less than the feces characteristic 7) (step S22). The determination can be determination, for example, as to whether there is no feces region other than “water” and “feces characteristic 7” in the legend in FIG. 6 in the classification image Img-r. The reason is that, a case where a region classified as feces characteristics 1 to 6 exist even in a part means that the pretreatment has not been completed.

When YES in step S22, the state confirmation apparatus 5 proceeds to determination of a result of a feces color analysis, and determines whether the feces color analysis result is any of “transparent” or “yellowish transparent”, and other than that (step S23). In a case of YES in step S23, the state confirmation apparatus 5 generates a determination result that preprocessing determination is an examination OK, assuming that a condition of the pretreatment determination is satisfied (step S24). Next, the state confirmation apparatus 5 transmits a notification (preprocessing determination notification) indicating a preprocessing determination result (herein, the examination OK) to at least one of the terminal apparatus of an examinee being a user of a toilet and the terminal apparatus 50 of a staff (step S25), and ends the processing. Of course, an order of the determination in steps S22 and S23 is not limited.

As a result, the examinee can know that it is in an examinable state, and can notify the staff of the fact. Alternatively, the staff can determine that the examinee is in a state where the examinee may perform the examination, and at a stage when an examination system for the examinee is ready, the staff can talk to the examinee. Particularly, with regard to the notification to the examiner, even when the notification is not made as character information, it is possible to save a time and effort for the examiner to view the character information by notifying the examiner by an automated voice using an intercom or the like.

On the other hand, in a case of NO in step S22 and in a case of NO in step S23, the state confirmation apparatus 5 generates a determination result that the preprocessing determination is an examination NG, assuming that the condition of the pretreatment determination is not satisfied (step S28). Next, the state confirmation apparatus 5 transmits the preprocessing determination notification indicating the examination NG to at least one of the terminal apparatus of the examinee and the terminal apparatus 50 of the staff (step S25), and ends the processing. Until the preprocessing determination notification indicating the examination OK is acquired, the examinee can perform excretion with an interval as needed, or the staff can prompt the examinee to excrete.

In addition, although not illustrated, the state confirmation apparatus 5 can also output an analysis result to the server 40 after the processing in step S24 and after the processing in step S28. The analysis result may also include a result of the preprocessing determination, but may also include the result of the preprocessing determination only when becoming the examination OK, for example. Note that, imaging data are basically not transmitted to the server 40 from a viewpoint of privacy and a viewpoint of reduction in an amount of transmission data, but may be transmitted to the server 40 on an assumption that only a person who has authority to manage the server 40 can access the imaging data.

As described above, in the present example embodiment, in addition to the effect described in the second example embodiment, for example, the following effect is achieved.

A first effect is a point that, by automatically determining a content of excrement identified by a combination of an optical camera and machine learning, it is possible to reduce variation of a determination criterion depending on a person (particularly, an examinee), which has been carried out so far.

A second effect is a point that a situation of pre-examination work of an examinee can be immediately recognized by notifying an event (sitting, excretion, foreign body detection, and the like) occurring in a toilet by a real time analysis, and thus an examiner is released from a situation where the examiner stays with an examinee during excretion. As a result, a time burden related to the examiner is reduced.

A third effect is a point that, when an analysis of an image captured by an optical camera is performed, all analysis processing is performed by a toilet sensor, and thus image data are not seen by a third party, and a mental burden related to privacy of an examinee is reduced.

A fourth effect is a point that, with the second and third effects, an examiner does not infringe on privacy of an examinee, and thus a mental burden as an opposite position is reduced.

A fifth effect is a point that improvement in criterion accuracy of pre-examination determination which has been carried out until now can be expected by performing determination by using a database recording an analysis result of excrement.

A sixth effect is a point that, since a pre-examination determination result can be remotely confirmed, even when an examinee has an infectious disease, a risk of infection to an examiner in pre-examination work can be avoided.

A seventh effect is a point that it can be attached to a toilet bowl (Western-style toilet bowl) having a general shape, can be produced and distributed as a single-type product, can be produced with low price, and can be carried.

### Fourth Example Embodiment

In the third example embodiment, it is assumed that an apparatus including the excrement analysis apparatus according to the first or second example embodiment is used as a state confirmation apparatus before colonoscopy, but the excrement analysis apparatus according to the first or second example embodiment may not be used. In a fourth example embodiment, an example in which state confirmation before colonoscopy is performed regardless of a classification method of a capturing target substance will be described. Since a component of a state confirmation apparatus according to the fourth example embodiment is the same as that of the state confirmation apparatus 5 described with reference to FIG. 11, and details of processing differ only in some of each of the components, the present example embodiment will be described with reference to FIG. 11, and FIGS. 2, 3, and the like. Note that, in the present example embodiment as well, basically, various examples applied in the third example embodiment that uses the first and second example embodiments can be applied except for a conflicting processing example.

As illustrated in FIG. 11, a state confirmation apparatus 5 according to the present example embodiment also includes an input unit 5a, a classification unit 5b, an output unit 5c, and a determination unit 5d, similarly to the state confirmation apparatus 5 according to the third example embodiment.

To briefly describe each unit, the input unit 5a inputs imaging data captured by an image capture apparatus being installed in such a way as to include, in a capturing range, an excretion range of excrement in a toilet bowl of a toilet. The classification unit 5b classifies a capturing target substance with respect to imaging data being input by the input unit 5a. The determination unit 5d determines whether a user of a toilet has completed pretreatment before colonoscopy, based on a classification result in the classification unit 5b. The output unit 5c outputs a determination result by the determination unit 5d as notification information to at least one of a colonoscopy staff observing a user of the toilet as an examinee of colonoscopy, and the examinee.

In addition, the state confirmation apparatus 5 according to the present example embodiment can also be adopted a configuration including a calculation unit described in the third example embodiment. The calculation unit calculates a feces amount being an amount of feces, based on a classification result by the classification unit 5b (particularly, a classification result by a second classification unit described later). For example, the calculation unit can calculate the feces amount, based on the classification result by the second classification unit described later. Note that, when it is not classified as feces, the feces amount can be calculated as zero. Then, the determination unit 5d can determine whether a user of the toilet has completed pretreatment, based on the classification result by the classification unit 5b and the feces amount calculated by a calculation unit 5e.

In addition, the state confirmation apparatus 5 according to the present example embodiment can also include a control unit (not illustrated) that controls the entire state confirmation apparatus 5 and a communication unit (not illustrated), and the control unit can include a part of the input unit 5a, the classification unit 5b, the output unit 5c, the determination unit 5d (, and the calculation unit) described above.

However, the classification unit 5b in the present example embodiment performs classification of excrement in the capturing target substance into any of feces, urine, and urine dripping, or any of feces, urine, feces+urine, and urine dripping, and also performs classification into a plurality of predetermined feces characteristics and classification into a plurality of predetermined feces colors.

In other words, the classification unit 5b according to the present example embodiment only needs to be able to perform classification of the capturing target substance in this manner, and semantic segmentation described in the first to third example embodiments may not be used at all or may be used only in a part. Hereinafter, an example in which the classification unit 5b performs primary classification (a primary analysis) and secondary classification (a secondary analysis), which will be described later, as classification processing, and uses the semantic segmentation only for the primary analysis will be described. However, the semantic segmentation may be used, for example, only for the secondary analysis, or may not be used for both of primary and secondary analyses.

Herein, although not illustrated, the classification unit 5b can include a first classification unit that performs a primary analysis and the second classification unit that performs a secondary analysis. Since the classification unit 5b also performs the secondary analysis after the primary analysis, it is assumed that the classification unit 5b includes a holding unit that temporarily holds imaging data to be analyzed until the secondary analysis. The holding unit may be a storage apparatus such as a memory.

The first classification unit classifies the capturing target substance into any of excrement, a foreign body not being allowed to be discarded into a toilet bowl 20, and another substance, and also classifies the excrement into any of feces, urine, and urine dripping, or any of feces, urine, feces+urine, and urine dripping. Also in the present example embodiment, the another substance can include at least one of a buttocks washing machine, toilet paper, and a substance after flushing excrement. The first classification unit can be performed in real time with acquisition of imaging data.

Then, when a classification result by the first classification unit becomes other than feces, the determination unit 5d in the present example embodiment determines that a user of the toilet has not completed the pretreatment. Therefore, when a determination result in the determination unit 5d indicates that pretreatment has not been completed, the output unit 5c can output, as notification information, information indicating that colonoscopy cannot be performed yet. In addition, the notification information output by the output unit 5c can include a classification result by the first classification unit. The notification information in this case may include information indicating the classification result, and may be predetermined information according to the classification result. For example, the notification information can be information that makes a notification that a foreign body is mixed when the foreign body is captured in imaging data. Particularly, the notification information can include a classification image being drawn by performing color classification, for each classification, of the classification result by the first classification unit. The classification image can be exemplified by, for example, a classification image Img-r in FIG. 6 or the like. In addition, excretion information being the classification result by the first classification unit may be output, as an output destination, to a server 40 that collects and manages the excretion information.

In addition, when the first classification unit classifies a capturing target substance into feces with respect to imaging data, the second classification unit classifies the capturing target substance into a plurality of feces characteristics and a plurality of feces colors. The second classification unit can perform classification, based on imaging data held in the holding unit, after the classification by the first classification unit, and can be performed in non-real time because more accuracy is required than processing of the first classification unit.

Further, when the classification result by the first classification unit becomes feces, the determination unit 5d in the present example embodiment determines whether a user of a toilet has completed pretreatment, based on a classification result by the second classification unit. In addition, when the classification result by the first classification unit is other than feces, the classification by the second classification unit may be stopped, and an excretion completion notification indicating that the pretreatment has not been completed may be made.

In addition, the notification information output by the output unit 5c can include a classification result by the second classification unit. The notification information in this case may include information indicating the classification result, and may be predetermined information according to the classification result. For example, the notification information can be information that makes a notification that there is a change in feces characteristic. Particularly, the notification information can include a classification image being drawn by performing color classification, for each classification, of the classification result by the second classification unit. The classification image can be exemplified by, for example, the classification image Img-r in FIG. 6 or the like. In addition, the excretion information being the classification result by the second classification unit may be output, as an output destination, to the server 40 that collects and manages the excretion information.

As described above, the state confirmation apparatus 5 divides an analysis of imaging data acquired from a camera into a primary analysis mainly for a purpose of notification requiring immediacy and a secondary analysis for a purpose of notification (and recording) not requiring immediacy. As a result, the state confirmation apparatus 5 can make a control unit such as a built-in CPU space saving and energy saving. This means that the state confirmation apparatus 5 efficiently uses a limited calculation resource by dividing the analysis processing into a function requiring immediacy and other functions. Further, the state confirmation apparatus 5 does not need to transmit imaging data acquired from the camera and other image data to an outside such as a cloud, and can perform an analysis of excrement only by its own apparatus installed in a toilet. In other words, all of an image and a video used for an analysis in the state confirmation apparatus 5 are processed in the state confirmation apparatus 5, and the image and the video are not transmitted to the outside. Therefore, it can be said that the state confirmation apparatus 5 has a configuration that leads to a reduction in a mental burden related to privacy of a user.

As described above, the state confirmation apparatus 5 can perform completion determination of pretreatment of colonoscopy without having to ask a user of a toilet while considering privacy of the user of the toilet. In addition, the state confirmation apparatus 5 can accurately collect information indicating a content of excrement excreted in a toilet bowl, and can also deal with a scene where an immediate notification to an observer is needed. In other words, the state confirmation apparatus 5 can achieve both of consideration for privacy of a user of a toilet, and notifying and recording, while improving installation of a sensor in a toilet for reducing a burden on excretion management in observing such as nursing care. The notifying and the recording herein are notifying of an immediate event at an observing site such as a caregiving site and recording of accurate information. Thus, the state confirmation apparatus 5 can reduce a physical/mental burden on an observer and a toilet user.

As described above, the state confirmation apparatus 5 can acquire an excretion start, foreign body detection, excrement detection, and excretion completion as a result of the primary analysis, and acquire a feces characteristic, a feces color, and a feces amount as a result of the secondary analysis. Any of the analysis results can be recorded in the server 40 on a cloud in a viewable state from a terminal apparatus 50, and it can also be configured in such a way as to transmit to the terminal apparatus 50. In addition, the server 40 can also be configured in such a way as to accumulate the received analysis result, perform a further analysis from the accumulated data, and then be able to notify to the terminal apparatus 50 of the analysis results or view from the terminal apparatus 50.

In addition, the state confirmation apparatus 5 or the present system including the state confirmation apparatus 5 can be used in a private house on an assumption that a user is one person, but preferably has a function of identifying a user on an assumption that a plurality of users are present. The function is as described by using face image data acquired by a second camera 15b or identification data acquired by a Bluetooth module 14b. As a result, it is possible to notify an examiner or an examinee of an entry notification, an exit notification, a sitting notification, a leaving notification, an excretion start notification, an excretion completion notification, a pretreatment determination notification, and the like together with a user name, and record detailed excretion information for each user in a clinical record.

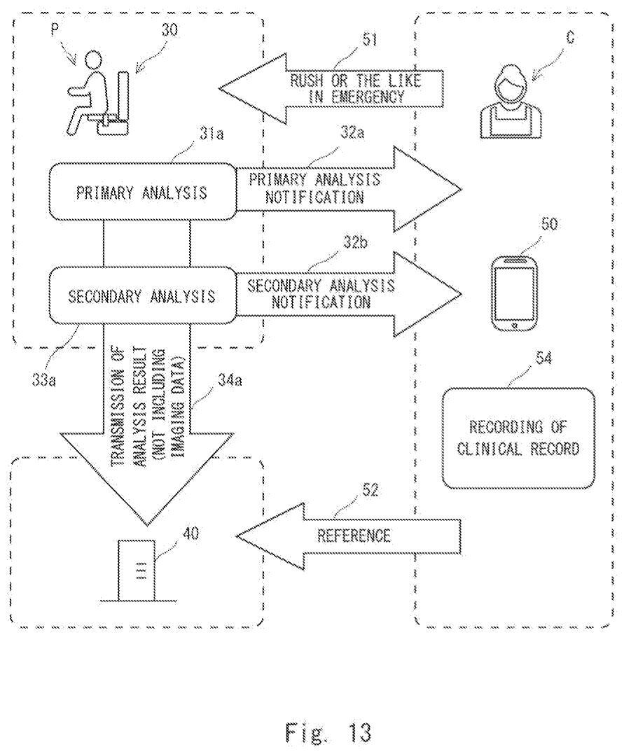

Next, with reference to FIGS. 13, 3, and 2, a real time analysis as a primary analysis (pre-analysis) and a non-real time analysis as a secondary analysis (the present analysis) will be schematically described. FIG. 13 is a schematic diagram for describing a processing example in the state confirmation apparatus 5.

In the present example embodiment, a second external box 11 includes the following device. The device is a device that performs a real time analysis as a primary analysis performed based on imaging data (image data) captured by a first camera 16b and a non-real time analysis as a secondary analysis performed based on the image data and a real time analysis result. In addition, the second external box 11 includes a communication device 14 that, under control of the device, notifies an examiner or an examinee and transmits an analysis result to the server 40 when an event occurs. A CPU 11a performs a real time analysis and a non-real time analysis while transmitting and receiving data to and from another portion via each of elements 11b, 11c, and 11d as necessary. Note that, in this example, the CPU 11a can also include a memory as an example of the holding unit.

As illustrated in FIG. 13, an example in which a user P uses a toilet bowl 30 with an analysis function being installed in a toilet, and an examiner C of the user P observes a state thereof is given. However, equipment 30 with the analysis function in the present example embodiment also has a determination function of the determination unit 5d. When the user P uses the toilet bowl 30 with the analysis function, the CPU 11a detects that the user sits on a toilet seat, based on a detection result from a range sensor 16a functioning as a sitting sensor. When detecting sitting, the CPU 11a instructs the first camera 16b to start capturing, and performs a primary analysis 31a, based on captured imaging data. The CPU 11a can perform foreign body determination and the like as the primary analysis 31a.

In a case where an immediate notification to an examiner due to foreign body detection and the like needs as a result of the primary analysis 31a, the CPU 11a transmits notification information (a primary analysis notification 32a) to the terminal apparatus 50 of the examiner C located at a place away from the toilet via a WiFi module 14a. In this way, the CPU 11a can transmit, to the terminal apparatus 50, foreign body information (foreign body information indicating a foreign body determination result) indicating whether a foreign body is included. The foreign body information is output as at least a part of the notification information. As a result, the examiner C is released from a situation where the examiner C accompanies (stays with) the user P being an examinee during excretion of the user P, and can also take a measure 51 by rushing and the like in case of emergency and log, to a clinical record, the fact that the examinee has started pre-examination work by the primary analysis notification 32a. Herein, the primary analysis notification 32a to be transmitted does not include imaging data.

The CPU 11a performs a secondary analysis 33a being a more detailed excrement analysis, based on imaging data and a primary analysis result, after the primary analysis 31a ends. Thus, the holding unit in the CPU 11a temporarily holds the primary analysis result as a part of secondary analysis target data. The CPU 11a performs transmission 34a of a secondary analysis result to the server 40 via the WiFi module 14a. In addition, the examiner C of the user P performs recording 54 of a clinical record of the user P while appropriately performing reference 52 to detailed excretion information of the user P being stored in the server 40, based on the received notification information, in the terminal apparatus 50.

In this way, the analysis results of the primary analysis 31a and the secondary analysis 33a are transmitted to the server 40 by the analysis result transmission 34a being performed by the communication function. The analysis result transmission 34a is transmitted without including the imaging data, but may be stored on a cloud as being accessible only by a person who has authority to manage a system as a use of learning data for future pretreatment determination. In parallel with the analysis result transmission 34a, a pretreatment determination result is transmitted to the terminal apparatus 50 as a secondary analysis notification 32b, and is recorded (logged) in the clinical record. Information recorded in the server 40 can also be used for performing generation 54 of a clinical record by an examiner or confirming a log by the examiner after the fact.

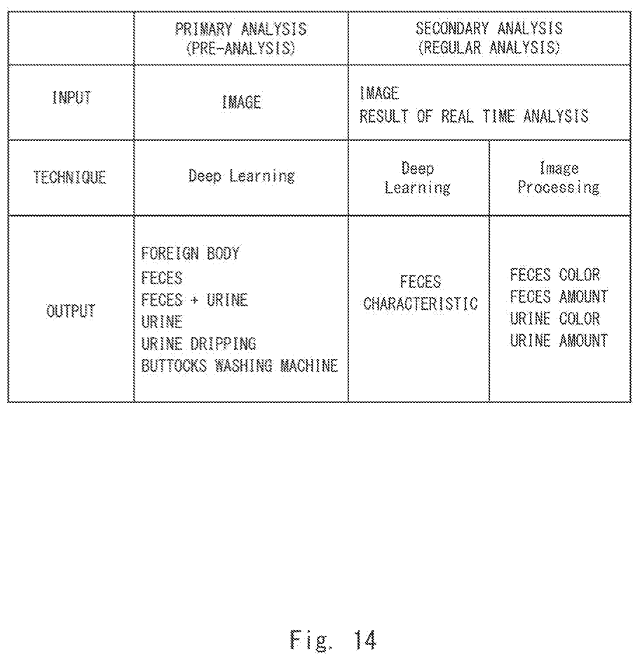

With reference to FIGS. 14 to 16, contents of the primary analysis 31a and the secondary analysis 33a will be described. FIGS. 14 to 16 are diagrams for describing a processing example in the state confirmation apparatus 5.

First, one example of an input, a technique, and an output of the primary analysis and the secondary analysis will be described with reference to FIG. 14. The primary analysis is an analysis requiring real time property, such as a notification to the examiner C. For the primary analysis, data (imaging data) of an image captured by the first camera 16b can be an input and be classified into any of the following six kinds by using semantic segmentation, for example, and a classification result can be an output. The six kinds are a foreign body (a diaper, a urine absorbing pad, and the like), feces, feces+urine, urine, urine dripping, and a buttocks washing machine.

Note that, the semantic segmentation can also be used for a comparison of an image (a background image) before excretion and the like with an image (an image during excretion or after excretion is completed) after the excretion. For example, a background image and an image after excretion are input as an input to a learning model, and which of the six kinds the image corresponds to can be output. Alternatively, a difference image of an image after excretion from a background image is acquired as preprocessing, the difference image is input to the learning model, and thus which of the six kinds the image corresponds to can be output. Note that, when it is classified into the buttocks washing machine, it can be determined that excretion is completed. The classification kinds are an example of a phenomenon being a trigger of a real time notification.

In this way, in the primary analysis, the notification information can be acquired from the imaging data by using a learned model that inputs the imaging data and outputs the notification information. The notification information can be, for example, predetermined information associated to a classification result of the notification information. As a result, in the state confirmation apparatus 5, information such as, for example, a start and completion of excretion, and contamination with a foreign body into excrement can be notified as the notification information to an examiner and the like, and the examiner and the like can acquire the information in real time. Note that, generation may be made by machine learning, regardless of an algorithm (machine learning algorithm), a hyper parameter such as the number of layers, and the like of the learned model. In addition, presence or absence of training data is not limited in the machine learning herein. However, in this example, it is assumed that a model that performs semantic segmentation is used as the learned model, and there are training data. In addition, the learned model used in the primary analysis may be plural, and for example, a different learned model can also be used between at least one kind of the six kinds described above and the other kinds.

In the secondary analysis, for example, an analysis can be performed by two techniques of DL and image processing (IP) with the imaging data from the first camera 16b and a primary analysis result as an input. For example, an analysis using DL can output a feces characteristic, and an analysis using IP can output a feces color, a feces amount, and a urine color. The semantic segmentation can also be used for an analysis of a feces characteristic. Note that, herein, the primary analysis is handled as preprocessing of the secondary analysis. In the secondary analysis, by using DL and IP, comparing an analysis result (which may be an image) acquired by performing the preprocessing with learned data is performed, and a feces characteristic, a feces color, and the like are output.

Herein, the DL technique can also be used for a comparison of an image (a background image) before excretion and the like with an image (an image during excretion or after excretion is completed) after the excretion. For example, a classification result in the primary analysis, a background image, and an image after excretion are input as an input to a learning model, and a feces characteristic can be output. Alternatively, a difference image of an image after excretion from a background image is acquired as preprocessing, a classification result in the primary analysis and the difference image are input to the learning model, and thus a feces characteristic can be output. In addition, only when a classification result in the primary analysis includes feces, an analysis by DL in the secondary analysis may be performed, and in that case, the classification result described above is unnecessary for an input to the learned model. In addition, a processing method in IP is not limited, and required detailed excretion information may be acquired. For example, matching processing with a comparison target image being stored in advance is performed by extracting a feature of an image, and the like, and a feces color and the like indicated by the comparison target image having a high matching rate can be output. Note that, in the secondary analysis, all outputs may be acquired by one of IP and DL.

In this way, in the secondary analysis, at least a part of the detailed excretion information can be acquired from the second analysis target data by using a learned model that inputs the second analysis target data (that may include a primary analysis result) and outputs the excretion information. Note that, generation may be made by machine learning, regardless of an algorithm (machine learning algorithm), a hyper parameter such as the number of layers, and the like of the learned model. In addition, presence or absence of training data is not limited in the machine learning herein. In addition, the learned model used in the primary analysis may be plural. Further, as described above, in the secondary analysis, at least a part of the detailed excretion information can be acquired by performing image processing on the second analysis target data. As described above, a method of the image processing and the like are not limited, and required detailed excretion information may be acquired.

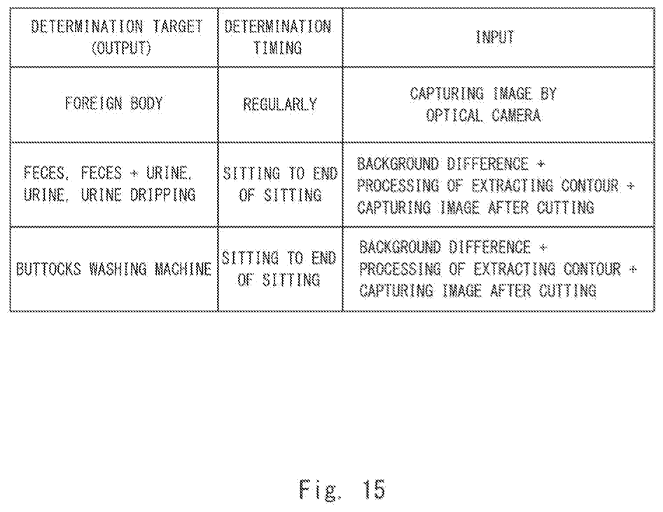

With reference to FIG. 15, a detailed example of the primary analysis is illustrated. In the primary analysis, a foreign body, a kind of excrement, and a buttocks washing machine can be set as a target of determination. First, foreign body detection is performed based on an image (imaging data) captured by the first camera 16b being an optical camera. The foreign body detection can be always performed, and an examiner is notified at a time of the foreign body detection. Subsequently, determination of feces, feces+urine, urine, and urine dripping with respect to excrement is performed by DL in a fixed cycle, based on a preprocessing image (and/or additional information) acquired by setting an image captured at timing of sitting as a background image, and then performing preprocessing on an image captured in a fixed cycle. The determination is performed until timing at which sitting ends. Herein, when a human body portion is detected, it is preferable to perform a process (mask process) of darkening as a non-analysis target in such a way as not to reflect a human body, an internal device, or the like being the non-analysis target in a background image. It is preferable that a similar mask process to that of the background image is also performed on an image captured at a fixed cycle after the background image is acquired. The additional information can include information such as a capturing date and time, and can also be, for example, information indicating a statistical value in consideration of the fixed cycle described above, information indicating an area such as dimensions, and the like. In addition, detection of a buttocks washing machine is also performed by a similar method at similar timing, and the determination of feces, feces+urine, urine, and urine dripping ends at timing when the buttocks washing machine is detected.

However, when the semantic segmentation is used in the primary analysis, for example, imaging data after the mask process are input, and thus classification thereof can be performed as single processing. On the other hand, as illustrated in FIG. 15, the primary analysis can perform classification of different targets according to the timing. In that case, it is possible to switch to an associated learned model according to the timing, and perform classification by using the learned model according to the timing (i.e., according to a classification target).

In this way, the CPU 11a may transmit, as a primary analysis result, at least one piece of information indicating a usage situation of a buttocks washing machine installed in a toilet bowl and information indicating that sitting is performed on the toilet bowl to the terminal apparatus 50 as at least a part of the notification information. As described above, the information indicating a usage situation of a buttocks washing machine can be acquired as a primary analysis result of imaging data. The reason is that a nozzle that ejects washing liquid or the washing liquid itself is included as a subject of the imaging data during use. In addition, the information indicating that sitting is performed on a toilet bowl can be acquired by a sitting sensor exemplified by the range sensor 16a. In this way, the primary analysis can also be performed by also using information other than the imaging data. Note that, a usage situation of a buttocks washing machine can also be recognized by the CPU 11a by, for example, being connected to the buttocks washing machine and acquiring information from that instead of an analysis of the imaging data.

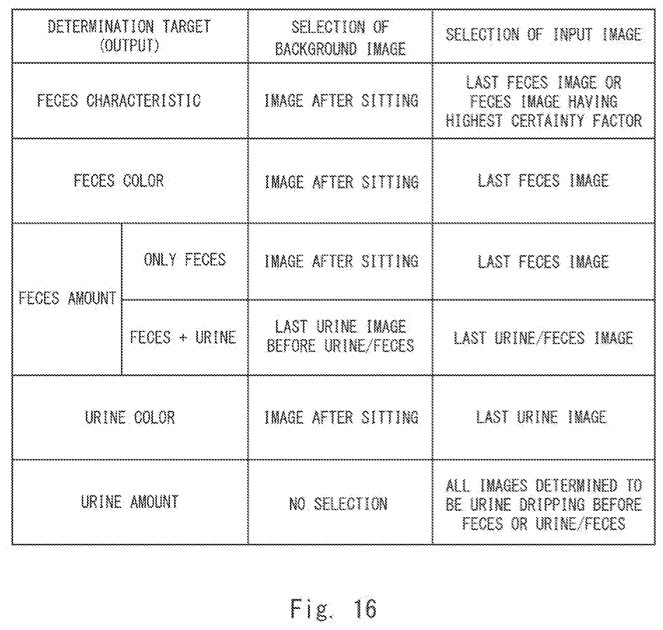

With referring to FIG. 16, a detailed example of the secondary analysis is illustrated. For the secondary analysis, an analysis can be performed on all targets being subjected to the preprocessing in the primary analysis by selection of a background image and an input image. The selection of a background image and an input image has combinations each suitable for a determination target, and thus a detailed analysis is performed. To describe a combination example, for a feces characteristic, first, an image after sitting is selected as a background image, and a last feces image is selected as an input image. In a case of only feces or a case of only urine, a target image is also similarly selected for a feces color, a feces amount, and a urine color. However, a urine image is used instead of feces for a urine color. In a case of feces+urine, a last urine image before urine/feces is used as a background image, and a last urine/feces image is used as an input image. Note that, an analysis of a urine amount can also be performed, and in that case, a background image is not used, and all images determined to be urine dripping are used as an input image.

Particularly, in the present example embodiment, as pretreatment determination, in order to acquire information indicating that there is no remaining feces in an intestine, determination that there is watery feces and a feces color is “yellowish transparent” or “transparent” is performed by using a feces characteristic, a feces color, and a feces amount as a determination material. Then, in the present example embodiment, by such determination, information on whether to perform an examination is acquired as a pretreatment determination result.

In this way, in the secondary analysis, the detailed excretion information can be output by identifying a feces characteristic and a feces color, and calculating a feces amount from acquired imaging data. In addition, in the secondary analysis, information indicating whether a feces amount and a urine amount subjected to threshold processing exceed a predetermined threshold value can be set as the detailed excretion information or added to the detailed excretion information. It is desirable that the detailed excretion information output as a result of the threshold processing is transmitted (notified) to the terminal apparatus 50 directly or via the server 40. With such a notification (may include an alert), an examiner can recognize a phenomenon needed to be handled.

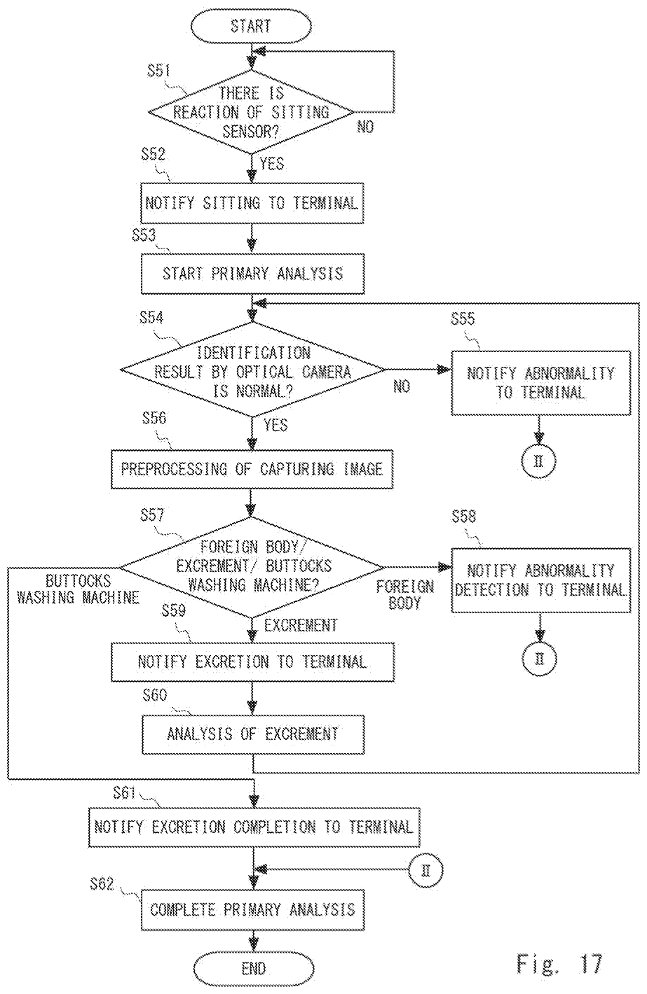

Next, one example of a procedure of primary analysis processing will be described with reference to FIG. 17. FIG. 17 is a flowchart for describing a processing example in the state confirmation apparatus 5, and is a flowchart illustrating one example of an operation content of a primary analysis in which a user entering a toilet and sitting on a toilet seat is a trigger. The operation content described herein can be mainly performed by the CPU 11a while the CPU 11a mainly controls each unit.

First, whether there is a reaction of the range sensor 16a functioning as a sitting sensor is checked (step S51). When there is no reaction in step S51 (in a case of NO), a reaction of the sitting sensor is waited. When an examinee as a user sits, the range sensor 16a reacts, and YES is determined in step S51. When YES is determined in step S51, sitting is notified to the terminal apparatus 50 (step S52), and the primary analysis also starts (step S53). Note that, when an entry is detected by a human detecting sensor 15a before sitting, the entry can also be notified to the terminal apparatus 50, and the same also applies to an exit.

In the primary analysis, the first camera 16b performs capturing inside a toilet bowl, and first, whether the inside of the toilet bowl can be normally identified is determined (step S54). When an abnormality is detected (in a case of NO in step S54), an abnormality notification is transmitted to at least one of the terminal apparatus 50 of an examiner and a terminal apparatus of the examinee (step S55). Herein, a case of being transmitted to the terminal apparatus 50 corresponds to a case where an examiner confirms pretreatment determination instead of an examinee, and the terminal apparatus of the examinee corresponds to a case where the examinee himself/herself performs the pretreatment determination, and the relationship is similar in subsequent processing. In this way, even when inside of the toilet bowl cannot be normally captured, it is preferable that notification information indicating the fact is transmitted to at least one of the terminal apparatus 50 of the examiner and the terminal apparatus of the examinee. On the other hand, when the inside of the toilet bowl can be normally identified (in a case of YES in step S54), the processing proceeds to a detailed analysis, and preprocessing of a captured image is performed first (step S56).

After the preprocessing of the captured image is performed in step S56, classification of a detection target object into a foreign body, excrement, and a buttocks washing machine is performed (step S57). When the foreign body is detected, a foreign body detection notification is made to the terminal apparatus 50 of the examiner (step S58). When the excrement is detected, an excretion notification (transmission of notification information indicating excretion) is made to at least one of the terminal apparatus 50 of the examiner and the terminal apparatus of the examinee (step S59), and an excrement analysis is also performed (step S60). By the excrement analysis, classification into any of feces, feces+urine, urine, and urine dripping is performed. After the processing of step S60, the processing returns to step S54.

When the detection target object detected in step S57 is the buttocks washing machine, excretion completion is determined, and an excretion completion notification (transmission of notification information indicating that excretion is completed) is made to at least one of the terminal apparatus 50 of the examiner and the terminal apparatus of the examinee (step S61). With the excretion completion notification in step S61, the primary analysis ends (step S62). In addition, the excretion completion notification may be transmitted only after a point in time when there is no reaction of the sitting sensor. The reason is that the buttocks washing machine may be used for twice or more. Note that, the primary analysis also ends after step S55 and after step S58.

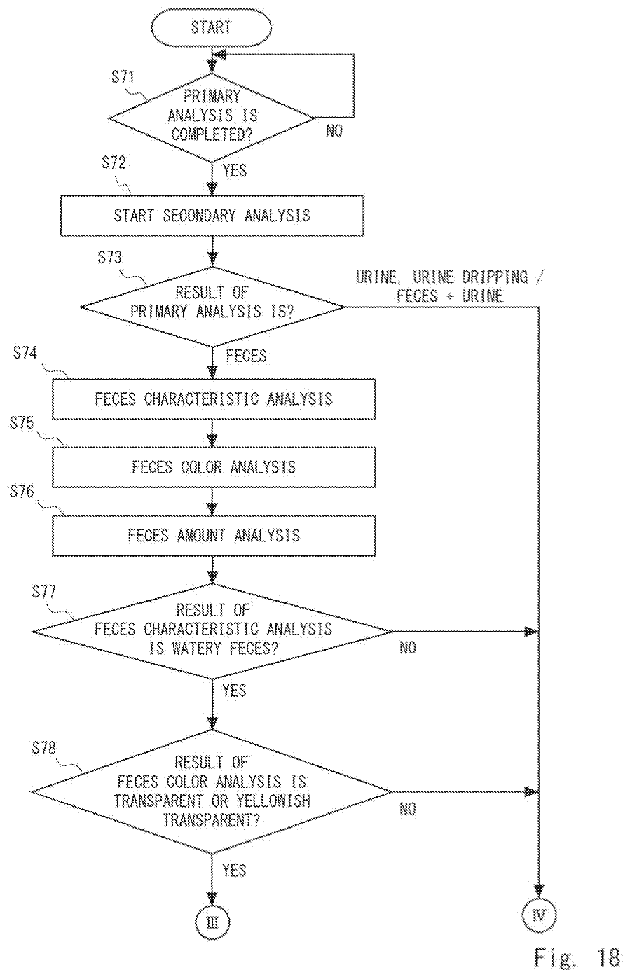

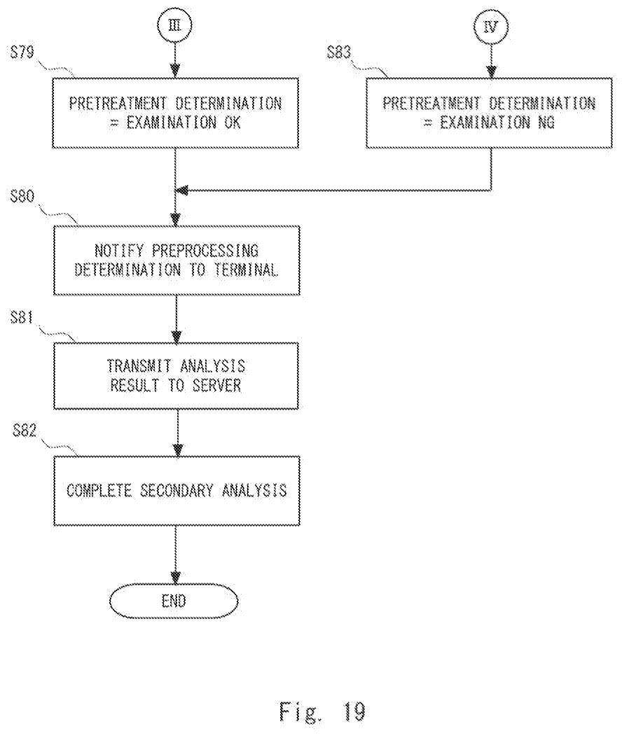

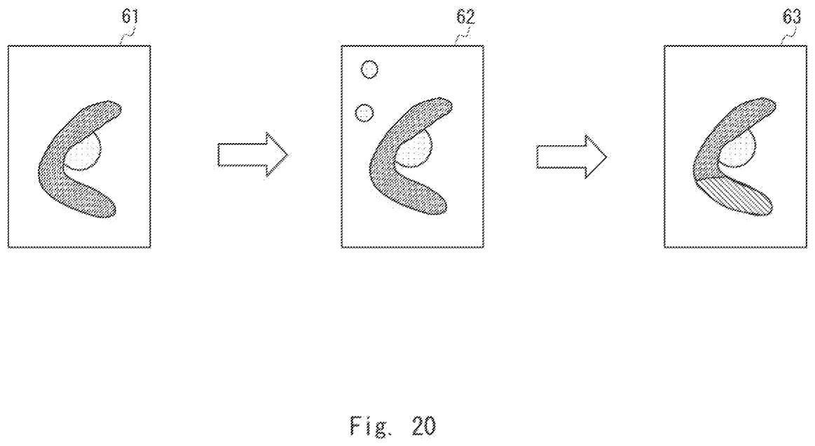

One example of a procedure of secondary analysis processing will be described with reference to FIGS. 18 to 20. FIGS. 18 and 19 are flowcharts for describing a processing example in the state confirmation apparatus 5, and are flowcharts illustrating one example of an operation content of the secondary analysis. The operation content described herein can be mainly performed by the CPU 11a while the CPU 11a mainly controls each unit. In addition, FIG. 20 is one example of a feces color analysis included in the secondary analysis in the processing example in FIG. 18. Note that, one example of a feces characteristic analysis will be described with reference to FIG. 7 again.

In the primary analysis illustrated in FIG. 17, while an analysis is performed by the space-saving and energy-saving CPU, a minimum necessary analysis in order to achieve a notification requiring immediacy to an examinee or an examiner is performed. In contrast, a more detailed analysis is performed on excrement in the secondary analysis.

First, whether the primary analysis is completed is determined (step S71), and, when the primary analysis is completed (in a case of YES), the secondary analysis starts (step S72). Alternatively, when a user identification function is provided, whether a predetermined excretion count is exceeded (or a predetermined period is elapsed) may be determined for each user, and the secondary analysis may start when the predetermined excretion count is exceeded.

An input of the secondary analysis and each analysis method can be as described with reference to FIG. 16, but first, a primary analysis result is determined (step S73), and different processing is performed according to the result.

When a result of determination of the primary analysis result in step S73 is urine, urine dripping, or feces+urine, pretreatment determination is set as an examination NG (not to be performed examination) with a reason in which the determination cannot be made properly because an object other than feces being a target of the pretreatment determination is mixed. Specifically, in this case, the state confirmation apparatus 5 generates a determination result that the preprocessing determination is the examination NG, assuming that a condition of the preprocessing determination is not satisfied (step S83). Next, the state confirmation apparatus 5 transmits a preprocessing determination notification indicating the examination NG to at least one of the terminal apparatus of the examinee and the terminal apparatus 50 of a staff (step S80), and proceeds to step S81. Until the preprocessing determination notification indicating an examination OK is acquired, the examinee can perform excretion with an interval as needed, or the staff can prompt the examinee to excrete.

When the primary analysis result in step S73 is feces, the feces characteristic analysis (step S74), the feces color analysis (step S75), and a feces amount analysis (step S76) are performed. Of course, an order of the analyses is not limited. Note that, when the primary analysis result in step S73 is urine or urine dripping, a urine color analysis can be performed, and a urine amount analysis can also be performed. In addition, each of the analyses in steps S74 to S76 may be performed by, for example, using each individual learning model, but a plurality of analyses or all of the analyses may also be performed by using one learning model.

Herein, in the feces characteristic analysis in step S74, an analysis is performed by a comparison with a learned image by DL by using an image having a highest degree of reliability. The image having a highest degree of reliability can be an image itself indicated by imaging data or an image acquired by performing preprocessing on imaging data by a preprocessing method suitable for an analysis of a feces characteristic. In addition, in the feces characteristic analysis, for example, an analysis can be performed in conformity to a Bristol stool scale illustrated in FIG. 7. By a result of the analysis, classification into any of types 1 to 7 as illustrated in FIG. 7 can be performed.

In addition, in the feces color analysis in step S75, for example, preprocessing as indicated in a processing procedure transitioning in an order of images 61, 62, and 63 in FIG. 20 can be performed. The preprocessing exemplified herein acquires the image 62 by removing a portion of a light color occupying a wide range from the original image 61, and then acquires the image 63 by removing a narrow region of the same color. Then, in the feces color analysis in step S75, an image like the image 63 in which necessary information is extracted (and/or added) by the preprocessing can be used, a range calculation between a color of the extracted feces and a feces reference color can be performed, and a color occupying a largest area in the extracted feces image can be set as a feces color. For example, feces-shaped objects of two colors are present in the image 63, and a color having a larger area can be set as a feces color. Note that, information added herein may also be, for example, information indicating an area, and the like.

Note that, when the urine color analysis is performed, the same method as that of the feces color analysis in step S75 can be adopted, but a target image is a urine image instead of a feces image, a range calculation to a reference color can be performed, and a color occupying a largest area can be set as a urine color.

In the feces amount analysis in step S76, a feces image (for example, the image 63, a primary analysis result, or the like) extracted by the preprocessing from an image at a point in time when excretion ends can used, and a feces amount can be calculated (estimated) as an area ratio in a fixed size. However, even with the same area, a feces amount varies by a feces characteristic, and may thus be calculated by an area ratio associated to the feces characteristic and a reference value of the feces amount.

When each of the analyses is completed, the state confirmation apparatus 5 determines whether an analysis result (classification result) of a feces characteristic is watery feces (e.g., “feces characteristic 7” in the legend in FIG. 6, or “water” in which a ratio of feces is less than the feces characteristic 7) (step S77). The determination can be determination, for example, as to whether there is no feces region other than “water” and “feces characteristic 7” in the legend in FIG. 6 in a classification image Img-r in FIG. 6. The reason is that, a case where a region classified as feces characteristics 1 to 6 exist even in a part means that the pretreatment has not been completed, that is, the condition of the pretreatment determination is not satisfied.

When YES in step S77, the state confirmation apparatus 5 proceeds to determination of a result of the feces color analysis, and determines whether a feces color analysis result is any of “transparent” or “yellowish transparent”, and other than that (step S78). In a case of YES in step S78, the state confirmation apparatus 5 generates a determination result that preprocessing determination is the examination OK, assuming that a condition of the pretreatment determination is satisfied (step S79). Next, the state confirmation apparatus 5 transmits a notification (preprocessing determination notification) indicating a preprocessing determination result (herein, the examination OK) to at least one of the terminal apparatus of an examinee being a user of a toilet and the terminal apparatus 50 of a staff (step S80). Of course, an order of the determination in steps S77 and S78 is not limited.

After the processing of step S80, the state confirmation apparatus 5 transmits an analysis result to the server 40 (step S81), and ends the processing. The analysis result may also include a result of the preprocessing determination, but may also include the result of the preprocessing determination only when becoming the examination OK, for example. Note that, imaging data are basically not transmitted to the server 40 from a viewpoint of privacy and a viewpoint of reduction in an amount of transmission data, but may be transmitted to the server 40 on an assumption that only a person who has authority to manage the server 40 can access the imaging data.

As a result, the examinee can know that it is in an examinable state, and can notify the staff of the fact. Alternatively, the staff can determine that the examinee is in a state where the examinee may perform the examination, and at a stage when an examination system for the examinee is ready, the staff can talk to the examinee. Particularly, with regard to the notification to the examiner, even when the notification is not made as character information, it is possible to save a time and effort for the examiner to view the character information by notifying the examiner by an automated voice using an intercom or the like.

On the other hand, in a case of NO in step S77 and in a case of NO in step S78, the state confirmation apparatus 5 generates a determination result that the preprocessing determination is the examination NG, assuming that the condition of the pretreatment determination is not satisfied (step S83). Next, the state confirmation apparatus 5 transmits the preprocessing determination notification indicating the examination NG to at least one of the terminal apparatus of the examinee and the terminal apparatus 50 of the staff (step S80). After the processing of step S80, the processing of step S81 is performed, and the processing ends. Until the preprocessing determination notification indicating the examination OK is acquired, the examinee can perform excretion with an interval as needed, or the staff can prompt the examinee to excrete.

In addition, in the present example embodiment, a system can be configured as a system that includes, as a toilet sensor, only an optical camera, a communication device, and the first classification unit, and performs another piece of processing by the server 40. The server 40 in the configuration example can include a reception unit, a second classification unit, a determination unit, and an output unit as follows. Hereinafter, the components will be briefly described, but basically, the second classification unit, the determination unit, and the output unit are similar to the units having the same names described with reference to FIGS. 13 to 20.

The reception unit receives a classification result acquired by performing first classification processing in the first classification unit, and receives imaging data when a classification result by the first classification processing indicates being classified into feces. The second classification unit in the configuration example classifies a capturing target substance into a plurality of predetermined feces characteristics and a plurality of predetermined feces colors with respect to the imaging data received by the receiving unit. The determination unit in the configuration example determines whether a user of a toilet has completed pretreatment before colonoscopy, based on a classification result by the second classification unit. The output unit in the configuration example outputs a determination result by the determination unit as notification information to at least one of a colonoscopy staff observing the user of the toilet as an examinee of colonoscopy, and the examinee.

Also in the configuration example, when the classification result received by the reception unit is other than feces, the determination unit can determine that a user of the toilet has not completed the pretreatment. In addition, when imaging data are received by the reception unit, the determination unit in the configuration example can determine whether a user of the toilet has completed the pretreatment, based on a classification result by the second classification unit.

In addition, in the present example embodiment, a system can be configured as a system that includes only an optical camera and a communication device as a toilet sensor, and performs another piece of processing by the server 40. The server 40 in the configuration example only needs to include the reception unit being capable of receiving imaging data, is equivalent to an example in which the state confirmation apparatus 5 is implemented by the server 40, differs only in a point of transmission/reception of information, and thus a detailed description thereof will be omitted.

As described above, in the present example embodiment, the first and the third to seventh effects described in the third example embodiment are achieved. In addition, in the present example embodiment, the following effect can be achieved in relation to the second effect described in the third example embodiment. In other words, in the present example embodiment, by notifying an event (sitting, excretion, foreign body detection, a pretreatment NG, and the like) occurring in a toilet by a first primary analysis, a situation of pre-examination work of an examinee can be immediately recognized. Therefore, also in the present example embodiment, an effect that an examiner is released from a situation where the examiner stays with an examinee during excretion, and a time burden related to the examiner is reduced is achieved.

### Other Example Embodiment

[a]

In each of the example embodiments, functions of each of apparatuses such as an excrement analysis apparatus, a server apparatus, and a state confirmation apparatus before colonoscopy, and each of apparatuses such as a terminal apparatus constituting a system together with each of the apparatuses have been described. Each of the apparatuses is not limited to an illustrated configuration example, and it is only necessary to achieve the function as the apparatus.

[b]

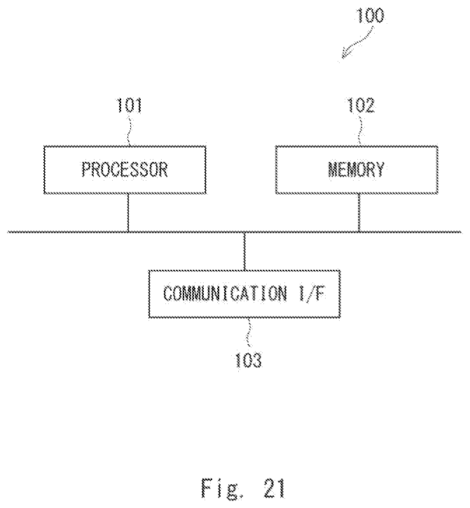

Each of the apparatuses described in the first to fourth example embodiments may have a following hardware configuration. FIG. 21 is a diagram illustrating one example of a hardware configuration of an apparatus. Note that, the same also applies to the another example embodiment [a] described above.

An apparatus 100 illustrated in FIG. 21 can include a processor 101, a memory 102, and a communication interface (I/F) 103. The processor 101 may be, for example, a micro processor, a micro processor unit (MPU), a CPU, or the like. The processor 101 may include a plurality of processors. The memory 102 is formed of, for example, a combination of a volatile memory and a nonvolatile memory. The functions of each of the apparatuses described in the first to fourth example embodiments are achieved by the processor 101 reading and executing a program stored in the memory 102. At this time, transmission/reception of information with another apparatus can be performed via the communication interface 103 or a not-illustrated input/output interface. Particularly, when the apparatus 100 is an excrement analysis apparatus or a state confirmation apparatus, transmission/reception of information (including imaging data) of an image capture apparatus built in or externally attached to the apparatus 100 can also be performed via the communication interface 103 or the not-illustrated input/output interface.

In the example described above, a program includes an instruction group (or a software code) that, when the program is loaded into a computer, cause the computer to execute one or more of the functions described in the example embodiments. The program may be stored in a non-transitory computer-readable medium or a tangible storage medium. By way of example, and not limitation, the computer-readable medium or the tangible storage medium includes a random-access memory (RAM), a read-only memory (ROM), a flash memory, a solid-state drive (SSD), or another memory technique, a CD-ROM, a digital versatile disc (DVD), a Blu-ray disk (registered trademark), or another optical disk storage, and a magnetic cassette, a magnetic tape, a magnetic disk storage, or another magnetic storage device. The program may be transmitted on a transitory computer-readable medium or a communication medium. By way of example, and not limitation, the transitory computer-readable medium or the communication medium includes a propagation signal in an electric, optical, acoustic, or another form.

Note that, the present disclosure is not limited to the example embodiment described above, and can be appropriately modified without departing from the spirit thereof. In addition, the present disclosure may be implemented by appropriately combining each of the example embodiments.

Some or all of the above-described example embodiments may be described as the following supplementary notes, but are not limited thereto.

### Supplementary Note 1

An excrement analysis apparatus including:
an input unit that inputs imaging data captured by an image capture apparatus being installed in such a way as to include, in a capturing range, an excretion range of excrement in a toilet bowl of a toilet;
a classification unit that performs classification of a capturing target substance in a pixel unit by using semantic segmentation with respect to imaging data being input by the input unit; and
an output unit that outputs a classification result by the classification unit.

### Supplementary Note 2

The excrement analysis apparatus according to supplementary note 1, wherein the classification unit classifies, for each pixel, the capturing target substance into any of the excrement, a foreign body not being allowed to be discarded into the toilet bowl, and another substance.

### Supplementary Note 3

The excrement analysis apparatus according to supplementary note 2, wherein the classification unit classifies the excrement into any of feces, urine, and urine dripping, or any of feces, urine, feces and urine, and urine dripping.

### Supplementary Note 4

The excrement analysis apparatus according to supplementary note 3, wherein the classification unit also performs together with at least one of classification of the feces into a plurality of predetermined feces characteristics, classification of the feces into a plurality of predetermined feces colors, and classification of the urine into a plurality of predetermined urine colors.

### Supplementary Note 5

The excrement analysis apparatus according to any one of supplementary notes 2 to 4, wherein the another substance includes at least one of a buttocks washing machine, toilet paper, and a substance after flushing the excrement.

### Supplementary Note 6

The excrement analysis apparatus according to supplementary note 5, wherein
the another substance includes at least the buttocks washing machine,
the classification unit stops subsequent classification processing when a classification result by the classification unit is classified into the buttocks washing machine, and
the output unit outputs an excretion completion notification to an observer observing a user of the toilet when a classification result by the classification unit is classified into the buttocks washing machine.

### Supplementary Note 7

The excrement analysis apparatus according to supplementary note 2, wherein
the output unit outputs an excretion notification to an observer observing a user of the toilet when a classification result by the classification unit is classified into the excrement,
after the excretion notification is output by the output unit, the classification unit classifies each pixel being classified into the excrement into any of feces, urine, and urine dripping, or any of feces, urine, feces and urine, and urine dripping, and also performs together with at least one of classification of the feces into a plurality of predetermined feces characteristics, classification of the feces into a plurality of predetermined feces colors, and classification of the urine into a plurality of predetermined urine colors, and
the output unit outputs a classification result of the feces, the urine, and the urine dripping, and at least one of a classification result of the feces characteristic, the feces color, and the urine color.

### Supplementary Note 8

The excrement analysis apparatus according to any one of supplementary notes 1 to 7, wherein the output unit outputs a classification result by the classification unit as information including a classification image drawn by performing color classification for each classification.

### Supplementary Note 9

The excrement analysis apparatus according to any one of supplementary notes 1 to 8, wherein the output unit notifies an observer observing a user of the toilet of a classification result by the classification unit.

### Supplementary Note 10

The excrement analysis apparatus according to any one of supplementary notes 1 to 9, further including a determination unit that determines whether a user of the toilet completes pretreatment before colonoscopy, based on a classification result by the classification unit, wherein
the classification unit also performs together classification of feces as the excrement into a plurality of predetermined feces characteristics and classification of the feces into a plurality of predetermined feces colors, and the output unit outputs a determination result by the determination unit as a classification result by the classification unit or as a part of a classification result by the classification unit.

### Supplementary Note 11

The excrement analysis apparatus according to supplementary note 10, further including a calculation unit that calculates a feces amount being an amount of the feces, based on a classification result by the classification unit, wherein the determination unit determines whether a user of the toilet completes the pretreatment, based on a classification result by the classification unit and the feces amount calculated by the calculation unit.

### Supplementary Note 12

A state confirmation apparatus before colonoscopy including:
an input unit that inputs imaging data captured by an image capture apparatus being installed in such a way as to include, in a capturing range, an excretion range of excrement in a toilet bowl of a toilet;
a classification unit that classifies a capturing target substance with respect to imaging data being input by the input unit;
a determination unit that determines whether a user of the toilet completes pretreatment before colonoscopy, based on a classification result by the classification unit; and
an output unit that outputs a determination result by the determination unit as notification information to at least one of a colonoscopy staff observing a user of the toilet as an examinee of colonoscopy, and the examinee,
wherein the classification unit classifies the excrement of the capturing target substance into any of feces, urine, and urine dripping, or any of feces, urine, feces and urine, and urine dripping, and also performs classification of the feces into a plurality of predetermined feces characteristics and classification of the feces into a plurality of predetermined feces colors.

### Supplementary Note 13

The state confirmation apparatus before colonoscopy according to supplementary note 12, wherein
the classification unit includes
a first classification unit that classifies the capturing target substance into any of the excrement, a foreign body not being allowed to be discarded into the toilet bowl, and another substance, and also classifies the excrement into any of feces, urine, and urine dripping, or any of feces, urine, feces and urine, and urine dripping, and
a second classification unit that classifies the capturing target substance into the plurality of feces characteristics and the plurality of feces colors with respect to the imaging data when the capturing target substance is classified into the feces by the first classification unit, and
the determination unit determines that a user of the toilet does not complete pretreatment when a classification result by the first classification unit becomes other than the feces, and determines whether a user of the toilet completes the pretreatment, based on a classification result by the second classification unit, when a classification result by the first classification unit becomes the feces.

### Supplementary Note 14

A state confirmation apparatus before colonoscopy including:
a reception unit that receives a classification result acquired by performing first classification processing of classifying, with respect to imaging data captured by an image capture apparatus being installed in such a way as to include, in a capturing range, an excretion range of excrement in a toilet bowl of a toilet, a capturing target substance into any of the excrement, a foreign body not being allowed to be discarded into the toilet bowl, and another substance, and also classifying the excrement into any of feces, urine, and urine dripping, or any of feces, urine, feces and urine, and urine dripping, and receives the imaging data when a classification result by the first classification processing indicates being classified into the feces;
a second classification unit that classifies the capturing target substance into a plurality of predetermined feces characteristics and a plurality of predetermined feces colors with respect to the imaging data received by the reception unit; a determination unit that determines whether a user of the toilet completes pretreatment before colonoscopy, based on a classification result by the second classification unit; and
an output unit that outputs a determination result by the determination unit as notification information to at least one of a colonoscopy staff observing a user of the toilet as an examinee of colonoscopy, and the examinee.

### Supplementary Note 15

The state confirmation apparatus before colonoscopy according to supplementary note 13 or 14, wherein the another substance includes at least one of a buttocks washing machine, toilet paper, and a substance after flushing the excrement.

### Supplementary Note 16

The state confirmation apparatus before colonoscopy according to any one of supplementary notes 13 to 15, wherein the notification information includes a classification result by the second classification unit.

### Supplementary Note 17

The state confirmation apparatus before colonoscopy according to supplementary note 16, wherein the notification information includes a classification image in which a classification result by the second classification unit is drawn by performing color classification for each classification.

### Supplementary Note 18

The state confirmation apparatus before colonoscopy according to any one of supplementary notes 13 to 17, further including a calculation unit that calculates a feces amount being an amount of the feces, based on a classification result by the second classification unit,
wherein the determination unit determines whether a user of the toilet completes the pretreatment, based on a classification result by the second classification unit and the feces amount calculated by the calculation unit.

### Supplementary Note 19

A state confirmation system before colonoscopy including:
an excrement analysis apparatus; and
a server apparatus configured to be connected to the excrement analysis apparatus, wherein
the excrement analysis apparatus includes
an input unit that inputs imaging data captured by an image capture apparatus being installed in such a way as to include, in a capturing range, an excretion range of excrement in a toilet bowl of a toilet,
a first classification unit that classifies, with respect to imaging data being input by the input unit, a capturing target substance into any of the excrement, a foreign body not being allowed to be discarded into the toilet bowl, and another substance, and also classifies the excrement into any of feces, urine, and urine dripping, or any of feces, urine, feces and urine, and urine dripping, and a transmission unit that transmits a classification result by the first classification unit to the server apparatus, and transmits the imaging data to the server apparatus when a classification result by the first classification unit indicates being classified into the feces, and
the server apparatus includes
a reception unit that receives a classification result by the first classification unit transmitted by the transmission unit, and receives the imaging data transmitted by the transmission unit when a classification result by the first classification unit indicates being classified into the feces,
a second classification unit that classifies, with respect to the imaging data received by the reception unit, the capturing target substance into a plurality of predetermined feces characteristics and a plurality of predetermined feces colors, a determination unit that determines whether a user of the toilet completes pretreatment before colonoscopy, based on a classification result by the second classification unit, and
an output unit that outputs a determination result by the determination unit as notification information to at least one of a colonoscopy staff observing a user of the toilet as an examinee of colonoscopy, and the examinee.

### Supplementary Note 20

The state confirmation system before colonoscopy according to supplementary note 19, wherein the determination unit determines that a user of the toilet does not complete pretreatment when a classification result received by the reception unit becomes other than the feces, and determines whether a user of the toilet completes the pretreatment, based on a classification result by the second classification unit, when the imaging data are received by the reception unit.

### Supplementary Note 21

The status confirmation system before colonoscopy according to supplementary note 19 or 20, wherein the another substance includes at least one of a buttocks washing machine, toilet paper, and a substance after flushing the excrement.

### Supplementary Note 22

An excrement analysis method including:
inputting imaging data captured by an image capture apparatus being installed in such a way as to include, in a capturing range, an excretion range of excrement in a toilet bowl of a toilet;
performing classification processing of classifying a capturing target substance in pixel unit by using semantic segmentation with respect to input imaging data; and outputting a classification result by the classification processing.

### Supplementary Note 23

The excrement analysis method according to supplementary note 22, wherein the classification processing classifies, for each of the pixels, the capturing target substance into any of the excrement, a foreign body not being allowed to be discarded into the toilet bowl, and another substance.

### Supplementary Note 24

The excrement analysis method according to supplementary note 23, wherein the classification processing classifies the excrement into any of feces, urine, and urine dripping, or any of feces, urine, feces and urine, and urine dripping.

### Supplementary Note 25

The excrement analysis method according to supplementary note 24, wherein the classification processing includes at least one piece of processing of classifying the feces into a plurality of predetermined feces characteristics, classifying the feces into a plurality of predetermined feces colors, and classifying the urine into a plurality of predetermined urine colors.

### Supplementary Note 26

The excrement analysis method according to any one of supplementary notes 23 to 25, wherein the another substance includes at least one of a buttocks washing machine, toilet paper, and a substance after flushing the excrement.

### Supplementary Note 27

The excrement analysis method according to any one of supplementary notes 22 to 26, further including determination processing of determining whether a user of the toilet completes pretreatment before colonoscopy, based on a classification result by the classification processing, wherein
the classification processing includes processing of performing classification of feces as the excrement into a plurality of predetermined feces characteristics and a plurality of predetermined feces colors, and
the outputting a classification result by the classification processing is that a determination result by the determination processing is output as a classification result by the classification processing or as a part of a classification result by the classification processing.

### Supplementary Note 28

The excrement analysis method according to supplementary note 27, further including calculation processing of calculating a feces amount being an amount of the feces, based on a classification result by the classification processing,
wherein the determination processing determines whether a user of the toilet completes the pretreatment, based on a classification result by the classification processing and the feces amount calculated by the calculation processing.

### Supplementary Note 29

A state confirmation method before colonoscopy including:
inputting imaging data captured by an image capture apparatus being installed in such a way as to include, in a capturing range, an excretion range of excrement in a toilet bowl of a toilet;
performing classification processing of classifying a capturing target substance with respect to input imaging data;
performing determination processing of determining whether a user of the toilet completes pretreatment before colonoscopy, based on a classification result by the classification processing; and
outputting a determination result by the determination processing as notification information to at least one of a colonoscopy staff observing a user of the toilet as an examinee of colonoscopy, and the examinee,
wherein the classification processing is processing of classifying the excrement of the capturing target substance into any of feces, urine, and urine dripping, or any of feces, urine, feces and urine, and urine dripping, and includes processing of also performing classification of the feces into a plurality of predetermined feces characteristics and classification of the feces into a plurality of predetermined feces colors.

### Supplementary Note 30

The state confirmation method before colonoscopy according to supplementary note 29, wherein
the classification processing includes
first classification processing of classifying the capturing target substance into any of the excrement, a foreign body not being allowed to be discarded into the toilet bowl, and another substance, and also classifying the excrement into any of feces, urine, and urine dripping, or any of feces, urine, feces and urine, and urine dripping, and
second classification processing of classifying the capturing target substance into the plurality of feces characteristics and the plurality of feces colors with respect to the imaging data when the capturing target substance is classified into the feces by the first classification processing, and
the determination processing determines that a user of the toilet does not complete pretreatment when a classification result by the first classification processing becomes other than the feces, and determines whether a user of the toilet completes the pretreatment, based on a classification result by the second classification processing, when a classification result by the first classification processing becomes the feces.

### Supplementary Note 31

The state confirmation method before colonoscopy according to supplementary note 30, wherein the another substance includes at least one of a buttocks washing machine, toilet paper, and a substance after flushing the excrement.

### Supplementary Note 32

A state confirmation method before colonoscopy including:
inputting, by an excrement analysis apparatus, imaging data captured by an image capture apparatus being installed in such a way as to include, in a capturing range, an excretion range of excrement in a toilet bowl of a toilet;
performing, by the excrement analysis apparatus, first classification processing of classifying, with respect to input imaging data, a capturing target substance into any of the excrement, a foreign body not being allowed to be discarded into the toilet bowl, and another substance, and also classifying the excrement into any of feces, urine, and urine dripping, or any of feces, urine, feces and urine, and urine dripping;
transmitting, by the excrement analysis apparatus, a classification result by the first classification processing to a server apparatus being connected to the excrement analysis apparatus, and transmitting the imaging data to the server apparatus when a classification result by the first classification processing indicates being classified into the feces;
receiving, by the server apparatus, a classification result by the first classification processing being transmitted from the excrement analysis apparatus, and receiving the imaging data transmitted from the excrement analysis apparatus when a classification result by the first classification processing indicates being classified into the feces;
performing, by the server apparatus, second classification processing of classifying, with respect to the received imaging data, the capturing target substance into a plurality of predetermined feces characteristics and a plurality of predetermined feces colors;
performing, by the server apparatus, determination processing of determining whether a user of the toilet completes pretreatment before colonoscopy, based on a classification result by the second classification processing; and
outputting, by the server apparatus, a determination result by the determination processing as notification information to at least one of a colonoscopy staff observing a user of the toilet as an examinee of colonoscopy, and the examinee.

### Supplementary Note 33

A state confirmation method before colonoscopy including:
receiving a classification result acquired by performing first classification processing of classifying, with respect to imaging data captured by an image capture apparatus being installed in such a way as to include, in a capturing range, an excretion range of excrement in a toilet bowl of a toilet, a capturing target substance into any of the excrement, a foreign body not being allowed to be discarded into the toilet bowl, and another substance, and also classifying the excrement into any of feces, urine, and urine dripping, or any of feces, urine, feces and urine, and urine dripping;
receiving the imaging data when a classification result by the first classification processing indicates being classified into the feces;
performing second classification processing of classifying the capturing target substance into a plurality of predetermined feces characteristics and a plurality of predetermined feces colors with respect to the received imaging data;
performing determination processing of determining whether a user of the toilet completes pretreatment before colonoscopy, based on a classification result by the second classification processing; and
outputting a determination result by the determination processing as notification information to at least one of a colonoscopy staff observing a user of the toilet as an examinee of colonoscopy, and the examinee.

### Supplementary Note 34

The state confirmation method before colonoscopy according to supplementary note 32 or 33, wherein the determination processing determines that a user of the toilet does not complete pretreatment when a received classification result becomes other than the feces, and determines whether a user of the toilet completes the pretreatment based on a classification result by the second classification processing, when the imaging data is received.

### Supplementary Note 35

A program for causing a computer to execute excrement analysis processing, the excrement analysis processing including:
inputting imaging data captured by an image capture apparatus being installed in such a way as to include, in a capturing range, an excretion range of excrement in a toilet bowl of a toilet;
performing classification processing of classifying a capturing target substance in a pixel unit by using semantic segmentation with respect to input imaging data; and
outputting a classification result by the classification processing.

### Supplementary Note 36

The program according to supplementary note 35, wherein the classification processing classifies, for each of the pixels, the capturing target substance into any of the excrement, a foreign body not being allowed to be discarded into the toilet bowl, and another substance.

### Supplementary Note 37

The program according to supplementary note 36, wherein the classification processing classifies the excrement into any of feces, urine, and urine dripping, or any of feces, urine, feces and urine, and urine dripping.

### Supplementary Note 38

The program according to supplementary note 37, wherein the classification processing includes at least one piece of processing of classifying the feces into a plurality of predetermined feces characteristics, classifying the feces into a plurality of predetermined feces colors, and classifying the urine into a plurality of predetermined urine colors.

### Supplementary Note 39

The program according to any one of supplementary notes 35 to 38, wherein the another substance includes at least one of a buttocks washing machine, toilet paper, and a substance after flushing the excrement.

### Supplementary Note 40

The program according to any one of supplementary notes 35 to 39, wherein
the excrement analysis processing includes determination processing of determining whether a user of the toilet completes pretreatment before colonoscopy, based on a classification result by the classification processing, the classification processing includes processing of performing classification of feces as the excrement into a plurality of predetermined feces characteristics and a plurality of predetermined feces colors, and
the outputting a classification result by the classification processing is that a determination result by the determination processing is output as a classification result by the classification processing or as a part of a classification result by the classification processing.

### Supplementary Note 41

The program according to supplementary note 40, wherein
the excrement analysis processing includes calculation processing of calculating a feces amount being an amount of the feces, based on a classification result by the classification processing, and
the determination processing determines whether a user of the toilet completes the pretreatment, based on a classification result by the classification processing and the feces amount calculated by the calculation processing.

### Supplementary Note 42

A program for causing a computer to execute state confirmation processing before colonoscopy, the state confirmation processing including:
inputting imaging data captured by an image capture apparatus being installed in such a way as to include, in a capturing range, an excretion range of excrement in a toilet bowl of a toilet;
performing classification processing of classifying a capturing target substance with respect to input imaging data;
performing determination processing of determining whether a user of the toilet completes pretreatment before colonoscopy, based on a classification result by the classification processing; and
outputting a determination result by the determination processing as notification information to at least one of a colonoscopy staff observing a user of the toilet as an examinee of colonoscopy, and the examinee,
wherein the classification processing is processing of classifying the excrement of the capturing target substance into any of feces, urine, and urine dripping, or any of feces, urine, feces and urine, and urine dripping, and includes processing of also performing classification of the feces into a plurality of predetermined feces characteristics and classification of the feces into a plurality of predetermined feces colors.

### Supplementary Note 43

The program according to supplementary note 42, wherein
the classification processing includes
first classification processing of classifying the capturing target substance into any of the excrement, a foreign body not being allowed to be discarded into the toilet bowl, and another substance, and also classifying the excrement into any of feces, urine, and urine dripping, or any of feces, urine, feces and urine, and urine dripping, and
second classification processing of classifying the capturing target substance into the plurality of feces characteristics and the plurality of feces colors with respect to the imaging data when the capturing target substance is classified into the feces by the first classification processing, and
the determination processing determines that a user of the toilet does not complete pretreatment when a classification result by the first classification processing becomes other than the feces, and determines whether a user of the toilet completes the pretreatment, based on a classification result by the second classification processing, when a classification result by the first classification processing becomes the feces.

### Supplementary Note 44

The program according to supplementary note 43, wherein the another substance includes at least one of a buttocks washing machine, toilet paper, and a substance after flushing the excrement.

### Supplementary Note 45

A program for causing a computer to execute state confirmation processing before colonoscopy, the state confirmation processing including:
receiving a classification result acquired by performing first classification processing of classifying, with respect to imaging data captured by an image capture apparatus being installed in such a way as to include, in a capturing range, an excretion range of excrement in a toilet bowl of a toilet, a capturing target substance into any of the excrement, a foreign body not being allowed to be discarded into the toilet bowl, and another substance, and also classifying the excrement into any of feces, urine, and urine dripping, or any of feces, urine, feces and urine, and urine dripping;
receiving the imaging data when a classification result by the first classification processing indicated being classified into the feces;
performing second classification processing of classifying the capturing target substance into a plurality of predetermined feces characteristics and a plurality of predetermined feces colors with respect to the received imaging data;
performing determination processing of determining whether a user of the toilet completes pretreatment before colonoscopy, based on a classification result by the second classification processing; and
outputting a determination result by the determination processing as notification information to at least one of a colonoscopy staff observing a user of the toilet as an examinee of colonoscopy, and the examinee.

### Supplementary Note 46

The program according to supplementary note 45, wherein the determination processing determines that a user of the toilet does not complete pretreatment when a received classification result becomes other than the feces, and determines whether a user of the toilet completes the pretreatment, based on a classification result by the second classification processing, when the imaging data is received.

Although the invention of the present application has been described with reference to the example embodiments, the invention of the present application is not limited to the above. Various modifications that can be understood by those skilled in the art can be made to the configuration and the details of the invention of the present application within the scope of the invention.

This application is based upon and claims the benefit of priority from Japanese patent application No. 2021-176986, filed on Oct. 28, 2021, the disclosure of which is incorporated herein in its entirety by reference.

### REFERENCE SIGNS LIST

1, 10 EXCREMENT ANALYSIS APPARATUS
1a, 5a INPUT UNIT
1b, 5b CLASSIFICATION UNIT
1c, 5c OUTPUT UNIT
5 STATE CONFIRMATION APPARATUS (STATE CONFIRMATION APPARATUS BEFORE COLONOSCOPY)
5d DETERMINATION UNIT
11 SECOND EXTERNAL BOX
11a CPU
11b CONNECTOR
11c, 11d USB I/F
12 INTER-BOX CONNECTION UNIT
13 FIRST EXTERNAL BOX
14a WiFi MODULE
14b BLUETOOTH MODULE
15a HUMAN DETECTING SENSOR
15b SECOND CAMERA
16a RANGE SENSOR
16b FIRST CAMERA
20 TOILET BOWL
21 MAIN BODY
22 TOILET SEAT
23 TOILET SEAT COVER
30 TOILET BOWL WITH EXCREMENT ANALYSIS APPARATUS
40 SERVER
41 CONTROL UNIT
42 STORAGE UNIT
50 TERMINAL APPARATUS
100 APPARATUS
10 PROCESSOR
102 MEMORY
103 COMMUNICATION INTERFACE

What is claimed is:

1. An excrement analysis apparatus comprising:
at least one memory storing instructions; and
at least one processor configured to execute the instructions to do excrement analysis, the excrement analysis includes:
inputting imaging data captured by an image capture apparatus being installed in such a way as to include, in a capturing range, an excretion range of excrement in a toilet bowl of a toilet;
performing classification processing of classifying a capturing target substance in a pixel unit by using semantic segmentation with respect to imaging data being input; and
outputting a classification result by the classification processing.

2. The excrement analysis apparatus according to claim 1, wherein the classification processing classifies, for each pixel, the capturing target substance into any of the excrement, a foreign body not being allowed to be discarded into the toilet bowl, and another substance.

3. The excrement analysis apparatus according to claim 2, wherein the classification processing classifies the excrement into any of feces, urine, and urine dripping, or any of feces, urine, feces and urine, and urine dripping.

4. The excrement analysis apparatus according to claim 3, wherein the classification processing also performs together with at least one of classification of the feces into a plurality of predetermined feces characteristics, classification of the feces into a plurality of predetermined feces colors, and classification of the urine into a plurality of predetermined urine colors.

5. The excrement analysis apparatus according to claim 2, wherein the another substance includes at least one of a buttocks washing machine, toilet paper, and a substance after flushing the excrement.

6. The excrement analysis apparatus according to claim 5, wherein
the another substance includes at least the buttocks washing machine,
the excrement analysis includes stopping subsequent classification processing when a classification result by the classification processing is classified into the buttocks washing machine, and
the outputting includes outputting an excretion completion notification to an observer observing a user of the toilet when a classification result by the classification processing is classified into the buttocks washing machine.

7. The excrement analysis apparatus according to claim 2, wherein
the outputting includes outputting an excretion notification to an observer observing a user of the toilet when a classification result by the classification processing is classified into the excrement,
after the excretion notification is output, the classification processing classifies each pixel being classified into the excrement into any of feces, urine, and urine dripping, or any of feces, urine, feces and urine, and urine dripping, and also performs together with at least one of classification of the feces into a plurality of predetermined feces characteristics, classification of the feces into a plurality of predetermined feces colors, and classification of the urine into a plurality of predetermined urine colors, and
the outputting includes outputting a classification result of the feces, the urine, and the urine dripping, and at least one of a classification result of the feces characteristic, the feces color, and the urine color.

8. The excrement analysis apparatus according to claim 1, wherein the output outputting includes outputting a classification result by the classification processing as information including a classification image drawn by performing color classification for each classification.

9. The excrement analysis apparatus according to claim 1, wherein the outputting includes notifying an observer observing a user of the toilet of a classification result by the classification processing.

10. The excrement analysis apparatus according to claim 1, wherein the excrement analysis further includes determining whether a user of the toilet completes pretreatment before colonoscopy, based on a classification result by the classification processing, wherein
the classification processing also performs together classification of feces as the excrement into a plurality of predetermined feces characteristics and classification of the feces into a plurality of predetermined feces colors, and
the outputting includes outputting a determination result by the determining as a classification result by the classification processing or as a part of a classification result by the classification processing.

11. The excrement analysis apparatus according to claim 10, wherein the excrement analysis further calculating a feces amount being an amount of the feces, based on a classification result by the classification processing, and
the determining includes determining whether a user of the toilet completes the pretreatment, based on a classification result by the classification processing and the feces amount calculated by the calculating.

12-21. (canceled)

22. An excrement analysis method comprising:
inputting imaging data captured by an image capture apparatus being installed in such a way as to include, in a capturing range, an excretion range of excrement in a toilet bowl of a toilet;
performing classification processing of classifying a capturing target substance in a pixel unit by using semantic segmentation with respect to input imaging data; and
outputting a classification result by the classification processing.

23. The excrement analysis method according to claim 22, wherein the classification processing classifies, for each of the pixels, the capturing target substance into any of the excrement, a foreign body not being allowed to be discarded into the toilet bowl, and another substance.

24. The excrement analysis method according to claim 23, wherein the classification processing classifies the excrement into any of feces, urine, and urine dripping, or any of feces, urine, feces and urine, and urine dripping.

25. The excrement analysis method according to claim 24, wherein the classification processing includes at least one piece of processing of classifying the feces into a plurality of predetermined feces characteristics, classifying the feces into a plurality of predetermined feces colors, and classifying the urine into a plurality of predetermined urine colors.

26. The excrement analysis method according to claim 23, wherein the another substance includes at least one of a buttocks washing machine, toilet paper, and a substance after flushing the excrement.

27. The excrement analysis method according to claim 22, further comprising determination processing of determining whether a user of the toilet completes pretreatment before colonoscopy, based on a classification result by the classification processing, wherein
the classification processing includes processing of performing classification of feces as the excrement into a plurality of predetermined feces characteristics and a plurality of predetermined feces colors, and
the outputting a classification result by the classification processing is that a determination result by the determination processing is output as a classification result by the classification processing or as a part of a classification result by the classification processing.

28. The excrement analysis method according to claim 27, further comprising calculation processing of calculating a feces amount being an amount of the feces, based on a classification result by the classification processing,
wherein the determination processing determines whether a user of the toilet completes the pretreatment, based on a classification result by the classification processing and the feces amount calculated by the calculation processing.

29-34. (canceled)

35. A non-transitory computer-readable medium storing a program for causing a computer to execute excrement analysis processing, the excrement analysis processing including:
inputting imaging data captured by an image capture apparatus being installed in such a way as to include, in a capturing range, an excretion range of excrement in a toilet bowl of a toilet;
performing classification processing of classifying a capturing target substance in a pixel unit by using semantic segmentation with respect to input imaging data; and
outputting a classification result by the classification processing.

36. The non-transitory computer-readable medium according to claim 35, wherein the classification processing classifies, for each of the pixels, the capturing target substance into any of the excrement, a foreign body not being allowed to be discarded into the toilet bowl, and another substance.

37-46. (canceled)
